(19) United States
(12) Patent Application Publication
(10) Pub. No.: US 2022/0193430 A1
(43) Pub. Date: Jun. 23, 2022

(54) ENHANCED DEFIBRILLATION SHOCK DECISIONS

(71) Applicant: Stryker Corporation, Kalamazoo, MI (US)

(72) Inventors: Fred W. Chapman, Newcastle, WA (US); Daniel W Piraino, Seattle, WA (US); Tyson G. Taylor, Bothell, WA (US); Robert G. Walker, Seattle, WA (US)

(21) Appl. No.: 17/560,043

(22) Filed: Dec. 22, 2021

Related U.S. Application Data
(60) Provisional application No. 63/130,223, filed on Dec. 23, 2020.

(51) Int. Cl. A61N 1/39; G16H 20/30; G16H 50/30

(52) U.S. Cl. CPC A61N 1/3925; A61N 1/39044; G16H 50/30; G16H 20/30; A61N 1/3993

(57) ABSTRACT

Defibrillators providing enhanced recommendations of whether to administer a defibrillation shock to patients are described. An example defibrillator determines an analysis factor, such as whether a patient has previously exhibited high-amplitude or “coarse” ventricular fibrillation (VF) during a particular time period. The defibrillator generates a shock index based on an electrocardiogram (ECG) of the patient and determines whether the patient is exhibiting a shockable rhythm by comparing the shock index to a threshold. The defibrillator generates the shock index and/or the threshold based on the analysis factor. The defibrillator outputs a recommendation based on the determination of whether the patient is exhibiting the shockable rhythm.

### CROSS-REFERENCE TO RELATED APPLICATION

This application claims the priority of U.S. Provisional Application No. 63/130,223, titled “ENHANCED DEFIBRILLATION SHOCK DECISIONS,” which was filed on Dec. 23, 2020 and is incorporated by reference herein in its entirety.

### BACKGROUND

Cardiac arrest is a condition in which an individual's heart ceases to function effectively. During cardiac arrest, the brain and other vital organs are unable to receive sufficient oxygenated blood, which can result in a sudden loss of consciousness. If untreated shortly after onset, cardiac arrest can result in long-term deficits or death. Thus, effective treatments must be applicable in a variety of environments where cardiac arrest is likely to occur, such as environments outside of hospitals or other specialized facilities for administering medical care.

Cardiopulmonary resuscitation (CPR) is a treatment that forces blood to vital organs using chest compressions, which can be administered manually or via a chest compression device, such as the LUCAS 3®, by Stryker Corporation of Kalamazoo, Mich. CPR is indicated for individuals experiencing cardiac arrest and can slow down damage to the vital organs by providing at least some blood flow despite the heart's disfunction. However, the underlying cause of the cardiac arrest is not treatable by CPR.

Some forms of cardiac arrest are the result of abnormal heart rhythms, such as ventricular fibrillation (VF) and pulseless ventricular tachycardia (V-tach). VF and pulseless V-tach are treatable by defibrillation, which is the delivery of an electrical shock to the heart. Because a defibrillation shock can be dangerous if administered to individuals without VF or pulseless V-tach, a medical device will generally identify and/or assist in the diagnosis of VF and pulseless V-tach based on electrocardiograms (ECGs). An ECG includes one or more lead signals that are indicative of the electrical activity of an individual's heart over time.

### BRIEF DESCRIPTION OF THE DRAWINGS

FIG. 1 illustrates an emergency environment in which a monitor-defibrillator is monitoring a patient that is experiencing cardiac arrest.

FIG. 2 illustrates an example ECG segment exhibiting high-amplitude VF.

FIG. 3 illustrates an example ECG segment exhibiting low-amplitude VF.

FIG. 4 is a diagram illustrating examples of possible shock index and threshold adjustments.

FIG. 5 illustrates an example process for determining a threshold based on an analysis factor and using the threshold to output a recommendation indicating whether a defibrillation shock is advised.

FIG. 6 illustrates an example process for determining a shock index based on an analysis factor and using the shock index to output a recommendation indicating whether a defibrillation shock is advised.

FIG. 7 illustrates an example process for determining whether a shockable rhythm is present in ECG data that includes a chest compression artifact.

FIG. 8 illustrates an example of an external defibrillator configured to perform various functions described herein.

### DETAILED DESCRIPTION

Various implementations described herein relate to systems, devices, and methods for adjusting, based on an analysis factor, a threshold for generating a recommendation of whether to administer a defibrillation shock to an individual. For instance, a medical device generates a shock index based on an ECG of the individual and compares the shock index to the threshold in order to determine whether a shockable rhythm (e.g., VF or V-tach) is present in the ECG. In various examples described herein, the medical device adjusts the threshold based on an analysis factor relating to, for example, whether the individual has previously exhibited coarse VF, whether the individual is a child (i.e., a pediatric patient), whether the individual is receiving chest compressions from a mechanical chest compression device, whether previously prompted CPR pause periods have been observed, whether the individual is or has exhibited a pulse, whether the individual is or has been pulseless, whether a change in a steepness of slopes in the ECG has changed greater than a threshold amount, or a combination thereof. The threshold adjustment improves the accuracy of the recommendation and/or reduces the chance of the medical device arriving at an indeterminate shock decision, in various cases.

Various implementations described herein relate to systems, devices, and methods for calculating, based on an analysis factor, a shock index used to determine whether to administer a defibrillation shock to an individual. For instance, a medical device generates a shock index based on an ECG of the individual and compares the shock index to the threshold in order to determine whether a shockable rhythm (e.g., VF or pulseless V-tach) is present in the ECG. In various examples described herein, the medical device generates the shock index based on an analysis factor relating to whether the individual has previously exhibited coarse VF, whether the individual is a child (i.e., a pediatric patient), whether the individual is receiving chest compressions from a mechanical chest compression device, whether previously prompted CPR pause periods have been observed, whether the individual is or has been pulseless, whether a change in a steepness of slopes in the ECG has changed greater than a threshold amount, shock indices based on previous ECG segments of the individual, or a combination thereof. The analysis factor improves the accuracy of the resultant recommendation, in various cases.

Implementations described herein solve specific problems in the technical field of medical devices. When an individual is observed in cardiac arrest, a rescuer will begin administering CPR. When the individual's condition is the result of a shockable arrhythmia, the shockable arrhythmia is generally diagnosed based on the ECG of the individual. However, the chest compressions administered to the individual cause a significant artifact in the ECG, making it difficult for the rescuer to identify the presence of the shockable arrhythmia in the ECG.

This issue is addressed, for example, by using analog and/or digital signal processing to remove the chest compression artifact from the ECG. In various examples, however, when the chest compression artifact is removed from the ECG, the shockable rhythm is nevertheless indiscernible to the rescuer. For example, the filtered ECG is difficult to evaluate. Thus, a medical device will independently analyze the ECG based on the filtered ECG. In some examples, the medical device generates a shock index based on the filtered ECG and compares the shock index to a shockable threshold. If the shock index is greater than the shockable threshold, the medical device concludes that the ECG of the individual includes a shockable rhythm. In some cases, the medical device further compares the shock index to a nonshockable threshold. If the shock index is less than the nonshockable threshold, the medical device concludes that the ECG of the individual does not include a shockable rhythm. In some examples in which the medical device is a monitor-defibrillator operating in a manual mode (or optionally, in an automatic mode), the medical device outputs a recommendation indicating whether a defibrillation shock is advised based on the presence or absence of the shockable rhythm. If the medical device detects the shockable rhythm, the medical device may charge the capacitor to help prepare for delivery of a defibrillation shock. In examples in which the medical device is an AED or any other type of defibrillator operating in an automatic mode, the medical device begins charging a capacitor in response to determining that the ECG of the individual includes the shockable rhythm and prompts the rescuer to administer the defibrillation shock.

In some cases, however, the medical device is unable to conclude whether the shockable rhythm is present or absent from the ECG. For example, if the shock index is less than the shockable threshold but greater than the nonshockable threshold, the medical device comes to an indeterminate decision about the ECG. In these cases, the medical device indicates that the rhythm in the ECG is indeterminate and/or prompts the rescuer to pause chest compressions so that the medical device can analyze the ECG without the chest compression artifact present. However, during such pauses, the brain and other vital organs of the individual may not be receiving sufficient oxygen and may be susceptible to long-term damage. In some examples in which the medical device is operating in manual mode, the device may optionally charge the defibrillation capacitor when an indeterminate decision is reached, readying the device to deliver a shock if the operator subsequently decides the rhythm is indeed shockable. The charge can readily be dumped from the capacitor (e.g., discharged without administering the charge to the individual) if the operator decides no shock is necessary, either by the operator manually dumping the charge or by the device automatically dumping charge from the capacitor after passage of a preset amount of time, e.g. 1 minute.

Various implementations described herein increase the likelihood that a medical device is able to conclude whether a shockable rhythm is present in an ECG. In various examples, the medical device considers one or more analysis factors in determining whether the shockable rhythm is present. The medical device adjusts and/or generates the thresholds and/or shock index based on these analysis factors. The analysis factors are, in some cases, parameters that are independent of the current ECG segment being analyzed. In various examples, the analysis factors are correlated with the presence or absence of a shockable rhythm in the individual's ECG. Thus, with the benefit of the analysis factors, the medical device is more likely to conclude that the individual is exhibiting the shockable rhythm or that the individual is not exhibiting the shockable rhythm. In some examples, the medical device uses an analysis factor to generate the shock index. In some implementations, the medical device uses an analysis factor to adjust the shockable or nonshockable thresholds for comparison with the shock index.

Particular examples will now be described with reference to the accompanying figures. The scope of this disclosure includes individual examples described herein as well as any combination of the examples, unless otherwise specified.

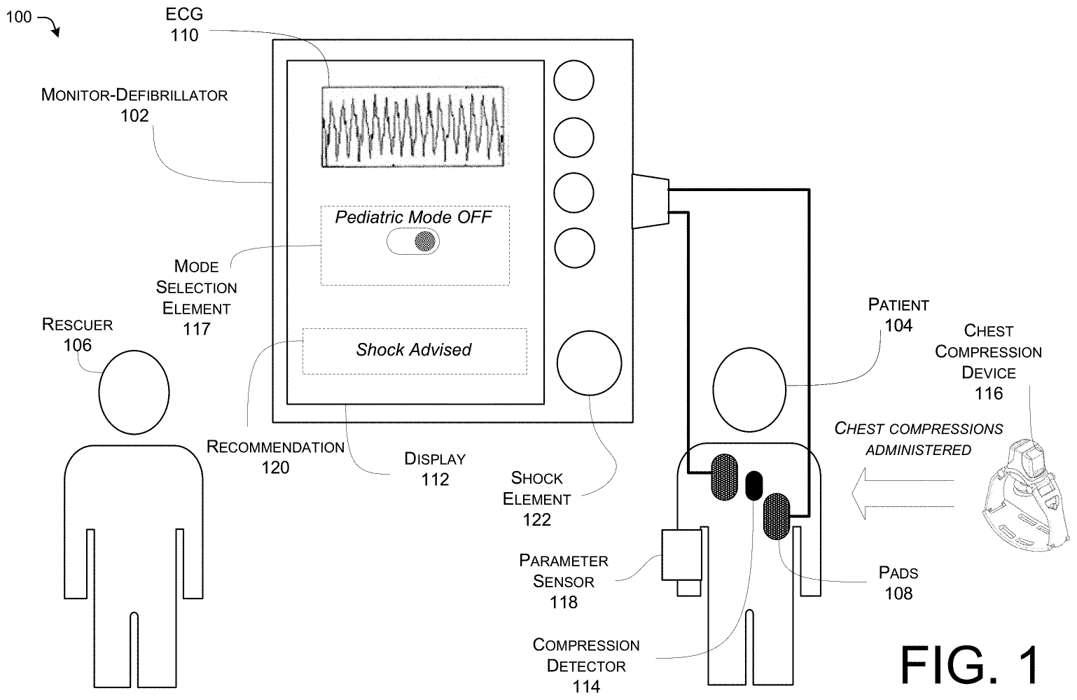

FIG. 1 illustrates an illustrates an emergency environment 100 in which a monitor-defibrillator 102 is monitoring a patient 104 that is experiencing cardiac arrest. The monitor-defibrillator 102 is operated by a rescuer 106, for instance. In some examples, the monitor-defibrillator 102 is an AED. The rescuer 106 has at least some medical training and is a trained operator of the monitor-defibrillator 102. For example, the rescuer is an emergency responder, a physician, a nurse, or the like. In some cases in which the monitor-defibrillator 102 is an AED, the rescuer 104 may be a lay responder outside the medical profession.

The monitor-defibrillator 102 includes pads 108 that are disposed on the patient 102. The pads 108 include multiple electrodes that are in contact with the patient 104. In some examples, the pads 108 are adhered to the skin of the patient 104. For example, the pads 108 are adhered to the skin of the patient 104 by a biocompatible adhesive. In various cases, the pads 108 include a substrate (e.g., a flexible substrate) that is adhered to the skin of the patient 104 by an adhesive.

The pads 108, for example, include therapeutic electrodes that are in contact with the patient 104. The monitor-defibrillator 102 is an external defibrillator, for instance, such that the therapeutic electrodes are in contact with the skin of the patient 102. The pads 108, for example, include two therapeutic electrodes, three therapeutic electrodes, ten therapeutic electrodes (e.g., enabling a 12-lead ECG), or thirteen therapeutic electrodes (e.g., enabling a 15-lead ECG). The therapeutic electrodes receive an electrical signal indicative of an electrical activity of the heart of the patient 104. For example, the heart of the patient 104 outputs an electrical field that impacts the relative voltages between the therapeutic electrodes.

The pads 108 are connected to additional circuitry in the monitor-defibrillator 102 by wired connections, wireless connections, or a combination thereof. In various examples, the monitor-defibrillator 102 includes a detection circuit configured to detect an ECG 110 of the patient 104 based on the relative voltages between the therapeutic electrodes. In some cases, the detection circuit includes an analog to digital converter that converts the relative voltages (representing the ECG 110 in an analog format) into digital data (representing the ECG 110 in a digital format). Although the ECG 110 pictured in FIG. 1 includes a single waveform corresponding to a single relative (lead) voltage between two or three therapeutic electrodes, implementations are not so limited.

The monitor-defibrillator 102 includes a display 112 that is configured to visually output information to the rescuer 106. In some examples, the display 112 includes a touchscreen configured to receive touch signals from the rescuer 106. These touch signals are examples of input signals that the monitor-defibrillator 102 receives from the rescuer 106. In various cases, the monitor-defibrillator 102 includes other types of input devices configured to receive input signals, such as buttons, knobs, and the like. In various examples, the monitor-defibrillator 102 outputs the ECG 110 on the screen. The rescuer 106, for instance, can assess a condition of the patient 104 based on the displayed ECG 110. In some examples, the rescuer 106 can determine whether the ECG 110 exhibits a shockable rhythm, such as VF or pulseless V-Tach. If the shockable rhythm is present in the ECG 110, the rescuer 106 may treat the shockable rhythm by causing the monitor-defibrillator 102 to administer a defibrillation shock to the patient 104.

However, as shown in FIG. 1, the monitor-defibrillator 102 detects the ECG 110 from the pads 108 as chest compressions are administered to the patient 104. The chest compressions are administered manually (e.g., by the rescuer) or by a chest compression device 116, such as LUCAS®, by Stryker Corporation of Kalamazoo, Mich. In some cases, the chest compression device 116 transmits, to the monitor-defibrillator 102, a signal indicative of the individual chest compressions, or indicating ongoing chest compressions, over a wired and/or wireless connection. The chest compressions can impart a significant artifact in the ECG 110. For example, the physical interface between the detection electrodes and the skin of the patient 104 are jostled by the chest compressions, the electrical impedance of the chest of the patient 104 varies with the chest compressions, the user or device administering the chest compressions impacts the voltage received by the detection electrodes, or a combination thereof. Due to the chest compression artifact (also referred to as a “compression artifact”) in the ECG 110, the rescuer 106 may be unable to accurately discern whether the patient 104 is exhibiting the shockable rhythm.

In various implementations, the monitor-defibrillator 102 analyzes the ECG 110 in order to determine whether the shockable rhythm is present. For example, the monitor-defibrillator 102 generates a filtered ECG segment by removing at least a portion of the chest compression artifact from a segment of the ECG 110. The segment is detected during a time period that is greater than or equal to 3 seconds and less than or equal to 30 seconds, for instance. The monitor-defibrillator 102 removes at least the portion of the chest compression artifact by applying an adaptive filter (e.g., a Wiener filter, a Kalman filter, or the like), applying a comb filter, applying an inverse comb filter, applying a high-pass filter, applying a band reject filter, applying a finite impulse response (FIR) filter, applying an infinite impulse response (IIR) filter, identifying and subtracting the chest compression artifact, or a combination thereof. In some cases, the monitor-defibrillator 102 converts the ECG 110 from the time domain into the frequency (e.g., a Fourier) domain, a Laplace domain, a Z-transform domain, or a wavelet (e.g., a continuous wavelet transform, a discrete wavelet transform, etc.) domain, and removes the chest compression artifact by analyzing the converted ECG 110.

In some implementations, the monitor-defibrillator 102 identifies the chest compression artifact by detecting the chest compressions administered to the patient 104. For instance, the monitor-defibrillator 102 determines a component of the ECG 110 segment that is time-aligned with the chest compressions. Because the chest compression artifact is time-aligned with the chest compressions administered to the patient 104, the monitor-defibrillator 102 identifies and removes the chest compression artifact from the ECG 110 based on the detected chest compressions.

According to some instances, the monitor-defibrillator 102 detects the chest compressions administered to the patient 104 by detecting an electrical impedance between the detection electrodes in the pads 108. This electrical impedance can be referred to as an electrical impedance of the patient 104, in some implementations. For example, the monitor-defibrillator 102 outputs a current (or voltage) across at least one detection electrode in a first one of the pads 108 and at least one detection electrode in a second one of the pads 108, detects a voltage (or current) at one of the detection electrodes, and determines the impedance of the patient 104 based on the voltage and the current (e.g., by dividing the voltage by the current). The impedance of the patient 104 changes over time based on the chest compressions administered to the patient 104. Thus, in some cases, the monitor-defibrillator 102 detects the chest compressions based on a waveform of the impedance of the patient 104 over time.

In some examples, the monitor-defibrillator 102 detects the chest compressions based on a compression detector 114 disposed on the patient 104. In various examples, the compression detector 112 generates a signal indicative of the chest compressions applied to the patient 104. In some instances, the compression detector 114 includes an accelerometer, a gyroscope, a pressure sensor, a multi-axial (e.g., tri-axial) magnetic sensor, or any combination thereof. The compression detector 114 transmits the signal that is indicative of the chest compression to the monitor-defibrillator 102 over a wired and/or wireless connection. In some implementations, the compression detector 114 generates multiple signals indicative of the chest compressions over time and transmits the signals to the monitor-defibrillator 102 (e.g., periodically), such that the monitor-defibrillator 102 detects the chest compressions substantially in real-time. In some cases, the monitor-defibrillator 102 detects the chest compressions based on the signal from the chest compression device 114.

The monitor-defibrillator 102 generates a shock index based on the filtered ECG segment. In various examples, the shock index corresponds to a likelihood and/or certainty that the filtered ECG segment includes the shockable rhythm and/or that the patient 104 exhibits the shockable rhythm during the time period corresponding to the segment of the ECG.

In some cases, the monitor-defibrillator 102 determines whether the shockable rhythm is present in the filtered ECG segment by comparing the shock index to a threshold. For example, the monitor-defibrillator 102 determines that the shockable rhythm is present (e.g., a “shockable” decision) if the shock index is greater than a first threshold and a second threshold, that the shockable rhythm is absent (e.g., a “nonshockable” decision) if the shock index is less than the first threshold and the second threshold, and that it is unclear whether the shockable rhythm is present (e.g., an “indeterminate” decision) if the shock index is greater than the first threshold and less than the second threshold.

According to various implementations described herein, the monitor-defibrillator 102 generates and/or adjusts the shock index based on an analysis factor associated with the patient 104. One example of an analysis factor is whether the chest compressions are being administered by the chest compression device 116 or manually (e.g., by the rescuer 106). In some implementations, the monitor-defibrillator 102 determines that the chest compression device 116 is administering the chest compressions based on a signal transmitted by the chest compression device 116 to the monitor-defibrillator 102. The signal, for instance, is transmitted over the wired and/or wireless connection between the monitor-defibrillator 102 and the chest compression device 116. In some cases, the monitor-defibrillator 102 determines that the chest compression device 116 is administering the chest compressions based on an input signal received from the rescuer 106 (e.g., at an input device of the monitor-defibrillator 102).

The shape of the chest compression artifact produced by the chest compression device 116 is, for example, similar to a nonshockable rhythm that includes QRS complexes. Thus, if the chest compressions are administered by the chest compression device 116, the monitor-defibrillator 102 analyzes the ECG 110 such that the shock index is more likely to distinguish between the QRS-like artifact generated by the chest compression device 116 and another nonshockable rhythm (e.g., asystole) or a shockable rhythm (e.g., VF or pulseless V-Tach without QRS complexes present) present in the ECG 110. In some cases, the chest compression artifact is similar to a shockable rhythm, such as VF or pulseless V-Tach. Thus, in some examples, if the chest compressions are administered by the chest compression device 116, then the monitor-defibrillator 102 analyzes the ECG 110 such that the shock index is more likely to distinguish between the shockable rhythm-like artifact generated by the chest compression device 116 and other heart rhythms present in the ECG 110. In various examples, the monitor-defibrillator 102 generates and/or adjusts the shock index based on whether the chest compressions are administered by the chest compression device 116 or manually by the rescuer 106. For instance, the monitor-defibrillator 102 is less likely to identify a nonshockable QRS rhythm in the ECG 110 when the chest compressions are administered by the chest compression device 116.

Another example of an analysis factor is whether the patient 104 has previously exhibited a shockable rhythm and/or what type of shockable rhythm was previously exhibited by the patient 104. In some instances, an ECG exhibiting nonshockable asystole is similar to an ECG exhibiting shockable, low-amplitude VF, which is also referred to as “fine VF.” The monitor-defibrillator 102, for instance, makes assumptions about whether asystole or low-amplitude VF is more likely to be observed and adjusts the shock index of the ECG 110 accordingly. For example, if the patient 104 is exhibiting high-amplitude VF, which is also referred to as “coarse VF,” the heart rhythm of the patient 104 is unlikely to transition to low-amplitude VF within a particular time period (e.g., 2 minutes). In some implementations, the monitor-defibrillator 102 generates and/or adjusts the shock index based on whether high-amplitude VF has been observed in the ECG 110 within the particular time period. For instance, the monitor-defibrillator 102 generates the shock index to be more likely to represent an indeterminate rhythm as nonshockable asystole when high-amplitude VF has been previously observed; and generates the shock index to be more likely to represent shockable low-amplitude VF when high-amplitude VF has not been previously observed in the ECG 110 of the patient 104.

In various examples, the monitor-defibrillator 102 detects high-amplitude VF and low-amplitude VF in the ECG 110. For instance, the monitor-defibrillator 102 determines that a segment of the ECG 110 includes VF and compares an amplitude of the segment to a threshold. In some cases, the monitor-defibrillator 102 detects amplitudes of peaks within the segment, averages the amplitudes, and compares the averaged amplitude to the threshold. In some specific examples, the threshold is between 0.15 mV to 0.3 mV, such as 0.2 mV. If the amplitude of the segment is greater than or equal to the threshold, the monitor-defibrillator 102 concludes that the segment includes high-amplitude VF. If, on the other hand, the amplitude is less than the threshold, the monitor-defibrillator 102 concludes that the segment includes low-amplitude VF. In some examples, the monitor-defibrillator 102 stores an indication of whether the VF in the segment is high-amplitude or low-amplitude, and uses the indication to generate the shock index of a later segment of the ECG 110. Alternatively or in addition, the amplitude measurement is used without classification as to low or high amplitude in order to generate the shock index of a later segment of the ECG 110.

A further example of an analysis factor is whether the patient 104 is an adult or a child. An ECG obtained from a pediatric patient may be less likely to exhibit noise or artifact (e.g., non-chest compression artifact) that would confound the analysis performed by the monitor-defibrillator 102 in generating the shock index. In some examples, the monitor-defibrillator 102 is more likely to generate the shock index to indicate a shockable or nonshockable decision, rather than an indeterminate decision, when the patient 104 is determined to be a child.

In some instances, the monitor-defibrillator 102 determines whether the patient 104 is an adult or a child based on a mode selection element 117. The mode selection element 117 is a user interface element configured to receive an input signal from the rescuer 106. The monitor-defibrillator 102 determines whether the patient 104 is an adult or a child based on the input signal. For instance, the mode selection element 117 includes a graphical user interface (GUI) element output by the display 110, such as a GUI slider. If the rescuer 106 slides the GUI element into a first position (e.g., by touching the display 110), the monitor-defibrillator 102 determines that the patient 104 is an adult (e.g., “Pediatric Mode OFF”). In contrast, if the rescuer 106 slides the GUI element into a second position, the monitor-defibrillator 102 determines that the patient 104 is a child (e.g., “Pediatric Mode ON”).

An example of an analysis factor includes a non-ECG physiological parameter of the patient 104. The physiological parameter is detected by a parameter sensor 118. In some examples, the parameter sensor 118 detects at least one of a blood pressure of the patient 104, an oxygenation (e.g., a peripheral capillary oxygen saturation (SpO₂)) of the patient 104, a capnograph (e.g., an end tidal CO₂) of the patient 104, a mechanical pulse (e.g., on a wrist) of the patient 104, an acceleration of any part of the patient 104, or a combination thereof. For example, the parameter sensor 118 includes a blood pressure sensor, an oxygenation sensor, a capnography sensor, a mechanical pulse sensor, an accelerometer, or a combination thereof. The parameter sensor 118 generates a signal indicative of the physiological parameter and transmits the signal to the monitor-defibrillator 102 over a wired and/or wireless connection. In various cases, the monitor-defibrillator 102 generates the shock index based on the physiological parameter of the patient 104.

In some examples, the monitor-defibrillator 102 determines whether the patient 104 is exhibiting or has exhibited a pulse. As used herein, the term “pulse” refers to a mechanical or chemical indication that a heart is pumping blood effectively and in a periodic fashion. The monitor-defibrillator 102 detects the pulse of the patient 104, for instance, based on the physiological parameter. If the patient 104 has exhibited a pulse within a particular time period (e.g., the last two minutes), the patient 104 is unlikely to be exhibiting low-amplitude VF. Thus, once the pulse has been detected, the monitor-defibrillator 102 generates the shock index to be more likely to reflect nonshockable asystole than low-amplitude VF, in some cases. In some cases, a transition from the VF being borderline high-amplitude (e.g., greater than a first threshold indicative of high-amplitude VF but less than a second threshold) and becomes borderline low-amplitude VF (e.g., less than the first threshold but greater than a third threshold) can occur in the span of a single CPR cycle or just through natural variation in VF amplitude. The monitor-defibrillator 102, in some examples, generates the shock index based on the last time a pulse of the patient 104 was present.

Another example of an analysis factor includes whether the patient 104 previously received chest compressions during a designated pause period. For example, the monitor-defibrillator 102 outputs an instruction (e.g., visually on the display 112) instructing the rescuer 106 to pause chest compressions to the patient 104. For instance, if the monitor-defibrillator 102 determines that the heart rhythm of the patient 104 is indiscernible from the ECG 110 (e.g., the monitor-defibrillator 102 arrives at an indeterminate decision), the monitor-defibrillator 102 outputs an instruction to pause the chest compressions so that the ECG 110 is detected without the chest compression artifact, thereby enhancing rhythm detection by the monitor-defibrillator 102. However, in examples in which the monitor-defibrillator 102 continues to detect chest compressions during the pause period, the monitor-defibrillator 102 generates the shock index based on the presence of those detected chest compressions. For example, since the ongoing chest compressions indicate that rescuer 106 is unlikely to follow future pauses, the monitor-defibrillator 102 generates the shock index to be less likely (or entirely unlikely) to indicate an indeterminate decision based on the ECG 110.

In some cases, the analysis factor includes slopes of the ECG 110 and/or rates of change of the slopes (positive slopes, negative slopes, or both) of the ECG 110 over time. For example, the monitor-defibrillator 102 determines a steepness of slopes in a first segment of the ECG 110 and a steepness of slopes in a different segment of the ECG 110. For example, the monitor-defibrillator 102 determines a derivative of the ECG 110 with respect to time and determines (e.g., local) minima and maxima in the derivative of the ECG 110. The steepness of slopes in a segment of the ECG 110 is indicative of whether the segment includes pulseless V-Tach, in some examples. For instance, the monitor-defibrillator 102 may determine whether the segment includes pulseless V-Tach by comparing the steepness values (e.g., the minima and maxima of the derivative of the ECG 110) to one or more thresholds. In some examples in which the steepness values are greater than a positive threshold and/or less than a negative threshold, the monitor-defibrillator 102 determines that the ECG 110 is exhibiting pulsatile V-Tach, which is a nonshockable rhythm. In contrast, if the monitor-defibrillator 102 determines that the steepness values are less than the positive threshold and/or greater than the negative threshold, the monitor-defibrillator 102 may determine that the ECG 110 is exhibiting pulseless V-Tach. This distinction is because the electrical conduction velocity through the heart of the patient 104 decreases as ischemia progresses, leading to less steep slopes over time when the patient 104 is not exhibiting a pulse. However, in an example in which the absolute values of the steepness values of the first segment are relatively low (e.g., under a predetermined threshold), the ECG 110 may be misclassified as nonshockable. To prevent misclassification, in some cases, the monitor-defibrillator 102 identifies both a global steepness threshold and a threshold relative to a first analyzed ECG segment (e.g., a segment classified as including shockable pulseless V-Tach). Alternatively or in addition, the monitor-defibrillator 102 identifies a steepness threshold based on second ECG segments obtained after the first ECG segment. In various examples, the monitor-defibrillator 102 uses any one of these thresholds to determine whether a third ECG segment exhibits pulseless V-Tach. In some implementations, if the monitor-defibrillator 102 determines that the steepness of the slopes has decreased between the time period of the first segment and the time period of a second segment, the monitor-defibrillator 102 generates the shock index to be more likely to indicate that a rhythm in the ECG 110 is shockable pulseless V-Tach, rather than a nonshockable rhythm.

In some examples, the analysis factor includes previously calculated shock indices. For instance, the monitor-defibrillator 102 generates shock indices for multiple segments of the ECG 110 that are detected in multiple time periods. The monitor-defibrillator 102 determines a trend or a range of the shock indices over the multiple time periods. In some examples, the monitor-defibrillator 102 calculates a new shock index of a new segment of the ECG 110 based on the trend and/or range of the previous segments of the ECG 110. In some cases, the monitor-defibrillator 102 determines that a shock index is outside of the trend or range, and instead of generating the recommendation 120 based on the shock index, the monitor-defibrillator 102 reevaluates the shock index based on another segment of the ECG 110.

According to various implementations described herein, the monitor-defibrillator 102 generates and/or adjusts the shockable and/or nonshockable thresholds based on an analysis factor associated with the patient 104. For example, the monitor-defibrillator 102 decreases the shockable and/or nonshockable thresholds based on determining that the chest compression device 116 is administering the chest compressions, rather than the rescuer 106. In some cases, the monitor-defibrillator 102 increases the shockable and/or nonshockable thresholds based on determining that the ECG 110 has exhibited high-amplitude VF within a particular time period. In some examples, the monitor-defibrillator 102 decreases the shockable threshold and/or increases the nonshockable threshold (e.g., narrows the indeterminate range) based on determining that the patient 104 is a child, rather than an adult. In some instances, the monitor-defibrillator 102 adjusts the shockable and/or nonshockable thresholds based on a physiological parameter of the patient 104. For example, the monitor-defibrillator 102 decreases the shockable and/or nonshockable thresholds based on determining that the patient 104 has exhibited a pulse within a particular time period. In some implementations, the monitor-defibrillator 102 decreases the shockable threshold and increases the nonshockable threshold based on determining that chest compressions were administered to the patient 104 during a previous pause period. In some examples, the monitor-defibrillator 102 decreases the shockable threshold and/or the nonshockable threshold based on determining that a steepness of slopes of the ECG has decreased over time. In some instances, the monitor-defibrillator 102 adjusts the shockable threshold and/or the nonshockable threshold based on a range and/or trend of shock indices corresponding to previous segments of the ECG.

By adjusting the shock index and/or the thresholds based on the analysis factor, the monitor-defibrillator 102 is able to more accurately identify whether the shockable rhythm is present in the ECG 110. The monitor-defibrillator generates a recommendation 120 based on the comparison between the shock index, the shockable threshold, and the nonshockable threshold. For instance, if the monitor-defibrillator 102 determines that the shock index is greater than the shockable threshold (and the nonshockable threshold), the monitor-defibrillator 102 generates the recommendation 120 to instruct the rescuer 106 to administer a defibrillation shock to the patient 104. If the monitor-defibrillator 102 determines that the shock index is less than the nonshockable threshold (and the shockable threshold), the monitor-defibrillator 102 generates the recommendation 120 to instruct the rescuer 106 to refrain from administering the defibrillation shock to the patient 104. If, however, the monitor-defibrillator 102 determines that the shock index is greater than the nonshockable threshold and less than the shockable threshold, then the monitor-defibrillator 102 generates the recommendation 120 to indicate that more time is needed to analyze the ECG 110 and/or to instruct the rescuer 106 to pause chest compressions for further analysis. However, by adjusting the shock index and/or the thresholds based on the analysis factor, in some cases, the monitor-defibrillator 102 is more likely to generate the recommendation 120 to instruct the rescuer 106 to administer or to refrain from administering the defibrillation shock, and less likely to generate the recommendation 120 to indicate that more time is needed to analyze the ECG and/or to instruct the rescuer 106 to pause the chest compressions.

In some examples, the recommendation 120 indicates a probability that the ECG 110 includes a shockable rhythm or a nonshockable rhythm to a user. For example, the monitor-defibrillator 102 determines that the ECG 110 has an a % likelihood of exhibiting a shockable rhythm (e.g., VF or pulseless V-tach) or determines that the ECG 110 has a b % likelihood of exhibiting a nonshockable rhythm (e.g., asystole, sinus rhythm, pulsatile V-tach, etc.). The medical device, for example, determines the a % or b % based on the comparison between the shock index and the threshold(s). In some examples, the recommendation 120 includes the probability without including an instruction of whether to administer the defibrillation shock to the patient 104.

In various cases, the rescuer 106 administers the defibrillation shock to the patient 104. For example, a shock element 122 of the monitor-defibrillator 102 receives an input signal from the rescuer 106. The shock element 122 includes any suitable input device. For example, the shock element 122 is a button. In some examples, the rescuer 106 provides the input signal in response to the recommendation 120 advising the rescuer 106 to administer the defibrillation shock. In some cases, the monitor-defibrillator 102 charges a capacitor and administers the defibrillation shock by discharging the capacitor to therapeutic electrodes in the pads 108. The monitor-defibrillator 102 begins charging the capacitor upon determining that the shockable rhythm (or an indeterminate rhythm) is present in the ECG 110, in response to the input signal at the shock element 122, or a combination thereof, for instance. Optionally, the monitor-defibrillator 102 begins charging the capacitor when it reaches an indeterminate decision. Thus, in some examples, the monitor-defibrillator 102 is ready to deliver a shock to the patient 104 if the rescuer 106 determines that the shock is warranted.

In some implementations, the monitor-defibrillator 102 averages or otherwise combines shock indices generated based on multiple segments of the ECG 110, and generates the recommendation 120 based on the average shock index. By relying on the average shock index, the monitor-defibrillator 102 reduces the risk of generating an erroneous recommendation 120 due to transient artifact within the ECG 110. According to particular cases, the average shock index is based on shock indices calculated based on three to five (overlapping and/or nonoverlapping) segments of the ECG 110. In some examples, a shock index fora more recent segment of the ECG 110 is weighted more heavily than a shock index for a less recent segment of the ECG 110 in the average shock index. By weighting the shock indices based on recency of the corresponding segments, the recommendation 120 can be rapidly updated based on sudden changes in the cardiac rhythm of the patient 104.

According to various implementations, if the monitor-defibrillator 102 is unable to generate the recommendation 120 to reflect a shock or no-shock decision within a threshold time, the monitor-defibrillator 102 outputs a prompt to at least temporarily cease chest compressions (e.g., the monitor-defibrillator 102 outputs a “stop CPR” message). For instance, the monitor-defibrillator 102 continuously and/or repeatedly analyzes the shock indices of segments of the ECG 110, but the shock indices remain in an indeterminate range. The threshold time, for example, is in a range of 10 seconds to 2 minutes, such as 10 seconds, 30 seconds, or 1 minute. In various cases, the monitor-defibrillator 102 detects that chest compressions have ceased (e.g., based on a signal detected by the compression detector 114). Upon determining that the chest compressions have ceased, in some examples, the monitor-defibrillator 102 generates a shock index without removing chest compression artifacts from the ECG 110, because chest compression artifacts are absent from the ECG 110. The cessation of chest compressions, in some cases, increases the chance that the monitor-defibrillator 102 generates a recommendation 120 to reflect a shock or no-shock decision. Thus, by limiting the amount of time that the monitor-defibrillator 102 analyzes the ECG 110 with chest compression artifacts present, the rescuer 106 is able to rapidly respond to a shockable rhythm exhibited by the patient 104 even when the monitor-defibrillator 102 is unable to discern the shockable rhythm in view of the chest compression artifacts.

In some implementations, the monitor-defibrillator 102 performs multiple (sometimes overlapping) analyses of the ECG 110 (or a segment(s) thereof). In these implementations, if a shock index exceeds the shock advised threshold by more than a threshold amount (e.g., a “very shockable” result), the timing of ECG segments analyzed can be shortened in order to rapidly output a recommendation 120 to indicate that treating the patient 104 with a defibrillation shock is advised. In some examples, this implementation is asymmetric. That is, if a shock index is below the no shock advised threshold by more than a threshold amount, this may not lead to a rapid output of a recommendation 120 to indicate that treating the patient 104 with a defibrillation shock is not advised. This is because there is no particular hurry in a nonshockable situation. Rather, in a nonshockable situation, the rescuer 106 may perform additional CPR.

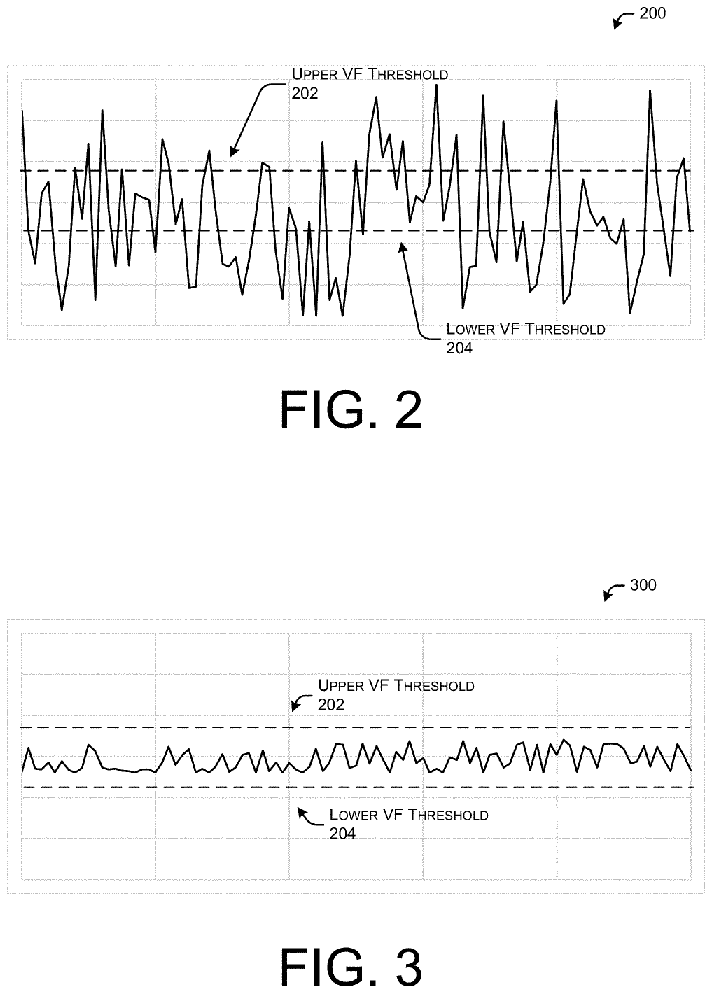

FIG. 2 illustrates an example ECG segment 200 exhibiting high-amplitude VF. Although unlabeled, the width of the boxes in FIG. 2 correspond to 1.0 second and the height of the boxes correspond to 0.5 mV, for instance. In some examples, the ECG segment 200 is a filtered segment derived based on an original ECG segment obtained from an individual receiving chest compressions.

In various cases, a medical device (such as the monitor-defibrillator 102 described above with respect to FIG. 1) determines that the ECG segment 200 is VF using any suitable method. For example, the medical device determines that a frequency of the ECG segment 200 corresponds to a VF frequency, a shape of the ECG segment 200 (in the time and/or frequency domains) is consistent with VF, or the like.

As illustrated in FIG. 2, the ECG segment 200 is compared to an upper VF 202 threshold and a lower VF threshold 204. The upper VF threshold 202 and the lower VF threshold 204 are represented as voltages. The upper VF threshold 202 is a positive voltage and the lower VF threshold 204 is a negative threshold. In some cases, the upper VF threshold 202 is equal to an absolute value of the lower VF threshold 204. For instance, the upper VF threshold is 0.3 mV and the lower VF threshold 204 is −0.3 mV. In some examples, the upper VF threshold 202 is 0.1 mV, 0.2 mV, or 0.3 mV. In some cases, the lower VF threshold 204 is −0.1 mV, −0.2 mV, or −0.3 mV.

The medical device determines that the VF is high-amplitude VF by comparing the ECG segment 200 to the upper VF threshold 202 and the lower VF threshold 204. For example, the medical device determines that at least a portion of the ECG segment 200 is greater than the upper VF threshold 202 and/or that at least a portion of the ECG segment 200 is less than the lower VF threshold 204. In some examples, the medical device calculates an average (e.g., an arithmetic mean and/or median) amplitude of multiple peaks in the ECG segment 200 and determines that the average amplitude is greater than the upper VF threshold 202. In some cases, the medical device calculates an average (e.g., an arithmetic mean and/or median) negative amplitude of multiple troughs in the ECG segment 200 and determines that the average negative amplitude is lower than the lower VF threshold 204.

Although FIG. 2 illustrates the ECG segment 200 with minimal offset, in some examples, the ECG segment 200 has a DC offset that shifts the ECG segment 200 vertically. To avoid misclassification of the VF due to an offset, in some examples, the medical device compares the peak-to-peak amplitude or a root mean squared (RMS) amplitude of the ECG segment 200 to a threshold. If the peak-to-peak amplitude or the RMS amplitude is greater than the threshold, the medical device classifies the ECG segment 200 as high-amplitude VF. If the peak-to-peak amplitude or the RMS amplitude is less than or equal to the threshold, the medical device classifies the ECG segment 200 as low-amplitude VF. The threshold for the peak-to-peak or RMS amplitude is, for example, 0.2 mV, 0.3 mV, 0.4 mV, 0.5 mV, or 0.6 mV.

In some implementations, the medical device stores an indication that the individual has high-amplitude VF. For example, the medical device stores a flag indicating that the individual has high-amplitude VF, a time at which the individual exhibited the high-amplitude VF, a time at which the medical device detected the high-amplitude VF, or a combination thereof. When the medical device evaluates a segment of the ECG obtained after the ECG segment 200, the medical device uses the stored data to generate a shock index for the latter ECG segment and/or to generate a threshold for determining whether a rhythm in the latter ECG segment is shockable, nonshockable, or indeterminate.

FIG. 3 illustrates an example ECG segment 300 exhibiting low-amplitude VF. Although unlabeled, the width of the boxes in FIG. 3 correspond to 1 second and the height of the boxes correspond to 0.5 mV, for instance. In some examples, the ECG segment 300 is a filtered segment derived based on an original ECG segment obtained from an individual receiving chest compressions.

In various cases, a medical device (such as the monitor-defibrillator 102 described above with respect to FIG. 1) determines that the ECG segment 300 includes VF using any suitable method. For example, the medical device determines that a frequency of the ECG segment 300 corresponds to a VF frequency, a shape of the ECG segment 300 (in the time and/or frequency domains) is consistent with VF, or the like.

As illustrated in FIG. 3, the ECG segment 300 is compared to the upper VF 202 threshold and the lower VF threshold 204. The medical device determines that the VF is low-amplitude VF by comparing the ECG segment 300 to the upper VF threshold 202 and the lower VF threshold 204. For example, the medical device determines that at least a portion (e.g., at least one peak) of the ECG segment 300 is less than the upper VF threshold 202 and/or that at least a portion (e.g., at least one trough) of the ECG segment 300 is greater than the lower VF threshold 204. In some examples, the medical device calculates an average (e.g., an arithmetic mean and/or median) amplitude of multiple peaks in the ECG segment 300 and determines that the average amplitude is less than the upper VF threshold 202. In some cases, the medical device calculates an average (e.g., an arithmetic mean and/or median) negative amplitude of multiple troughs in the ECG segment 300 and determines that the average negative amplitude is greater than the lower VF threshold 204.

In some implementations, the medical device stores an indication that the individual has low-amplitude VF. For example, the medical device stores a flag indicating that the individual has low-amplitude VF, a time at which the individual exhibited the low-amplitude VF, a time at which the medical device detected the low-amplitude VF, or a combination thereof. When the medical device evaluates a segment of the ECG obtained after the ECG segment 300, the medical device uses the stored data to generate a shock index for the latter ECG segment and/or to generate a threshold for determining whether a rhythm in the latter ECG segment is shockable, nonshockable, or indeterminate.

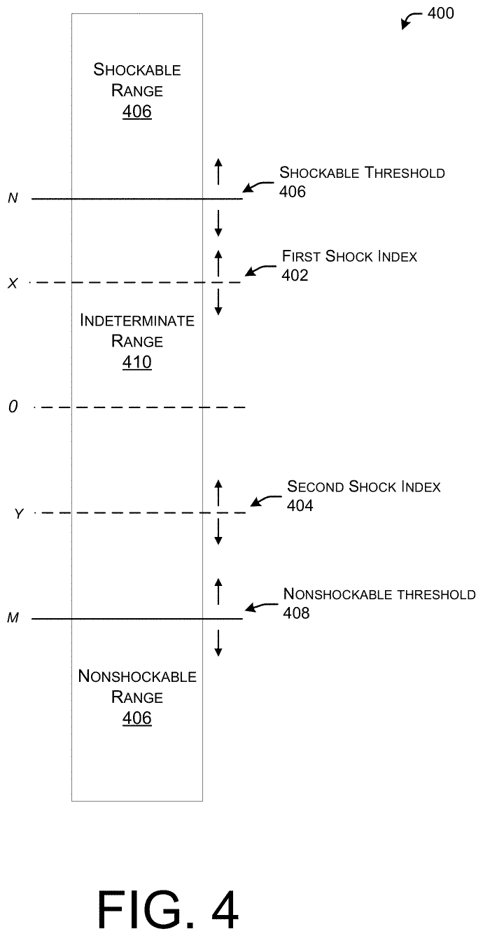

FIG. 4 is a diagram 400 illustrating examples of possible shock index and threshold adjustments. In various examples, a medical device, such as the monitor-defibrillator 102 described above with reference to FIG. 1, calculates a shock index of an individual based on a computing model (e.g., a regression) model that accepts various ECG features and/or other analysis factors as inputs and provides a shock index as an output.

In various implementations, a medical device (such as the monitor-defibrillator 102 described above with reference to FIG. 1) calculates a shock index (e.g., a first shock index 402 or a second shock index 404) of an ECG segment of an individual based on the computing model. In various examples, the medical device calculates and/or adjusts the shock index based on one or more analysis factors. These analysis factors, in some implementations, change the position of the shock index in the diagram 400. Examples of analysis factors include whether the ECG of the individual previously exhibited high-amplitude VF within a particular time period, whether the individual is a child or an adult, a non-ECG physiological parameter of the individual, whether the individual has exhibited a pulse within a particular time period, whether chest compressions have been administered during a pause period, whether steepnesses of slopes in the ECG have decreased over time, based on a range and/or trend of shock indices corresponding to previous segments of the ECG, or a combination thereof.

For example, the medical device determines the shock index based on determining that a chest compression device is administering chest compressions to the individual, rather than a human rescuer. In some cases, the medical device determines the shock index based on determining that the ECG has exhibited high-amplitude VF within a particular time period. In some examples, the medical device determines the shock index based on determining that the individual is a child, rather than an adult. In some instances, the medical device determines the shock index based on a physiological parameter of the individual. In some implementations, the medical device determines the shock index based on determining that chest compressions were administered to the individual during a previous pause period. In some examples, the medical device determines the shock index based on determining that a steepness of slopes of the ECG has decreased over time. In some instances, the monitor-defibrillator 102 determines the shock index based on a range and/or trend of shock indices corresponding to previous segments of the ECG. In some cases, the medical device determines the shock index based on whether the medical device has previously administered a shock to the individual (e.g., within a particular time period, such as a five-minute time period ending when the medical device determines the shock index).

In various examples, the medical device determines whether to decide and/or recommend administration of a defibrillation shock to the individual based upon a comparison between the shock index and a shockable threshold 406 and a comparison between the shock index and a nonshockable threshold 408. In some examples, the shockable threshold 406 and the nonshockable threshold 408 are derived based on a pre-specified certainty. For example, the shockable threshold 406 and/or the nonshockable threshold 406 correspond to a particular probability that a positive shock index indicates a shockable rhythm or a negative shock index indicates a nonshockable rhythm. The probability is, for instance, between 80% and 99%. In some cases, the medical device receives an input signal indicative of the probability.

In some implementations, the medical device outputs a signal indicative of the probability to a user. For example, the medical device determines that the ECG has an a % likelihood of exhibiting a shockable rhythm (e.g., VF or pulseless V-tach) or determines that the ECG has a b % likelihood of exhibiting a nonshockable rhythm (e.g., asystole, sinus rhythm, pulsatile V-tach, etc.). The medical device, for example, determines the a % or b % based on the comparison between the shock index and the shockable threshold 406 or based on the comparison between the shock index and the nonshockable threshold 408. In some examples, the medical device communicates the a % probability or the b % probability to the user. For instance, the medical device outputs a visual signal or an audible signal indicative of the a % probability or the b % probability by a screen or a speaker.

If the shock index of the individual is greater than the shockable threshold 406, for instance, the medical device determines that the ECG segment includes a shockable rhythm (e.g., VF or pulseless V-tach) and a defibrillation shock is indicated. For example, if the first shock index 402 is generated based on the ECG segment, the first shock index 402 is equal to X, the shockable threshold 406 is equal to N, and X>N, then the medical device determines that the ECG segment is indicative of a shockable rhythm. If the shock index of the individual is less than the nonshockable threshold 408, then the medical device determines that the ECG segment includes a nonshockable rhythm (e.g., asystole, a rhythm including QRS complexes, etc.). For example, if the second shock index 404 is generated based on the ECG segment, the second shock index 404 is equal to Y, the nonshockable threshold 408 is equal to M, and M>Y, then the medical device determines that the ECG segment is indicative of a nonshockable rhythm.

An indeterminate range 410 is defined between the shockable threshold 406 and the nonshockable threshold 408. If the shock index of the individual is within an indeterminate range 410, such that the shock index is greater than the nonshockable threshold 408 and less than the shockable threshold 406, then the medical device is unable to determine, with sufficient certainty, whether the ECG segment includes a shockable rhythm or a nonshockable rhythm. For example, if the first shock index 402 is generated based on the ECG segment, the first shock index 402 is equal to X, the shockable threshold 406 is equal to N, the nonshockable threshold 408 is M, and N>X>M, then the medical device determines that the ECG segment is indeterminate. In some examples, the medical device outputs a recommendation based on whether the ECG segment includes the shockable rhythm, the nonshockable rhythm, or is indeterminate.

In some examples, the medical device adjusts the shockable threshold 406 and/or the nonshockable threshold 408 based on an analysis factor. For example, the medical device adjusts the shockable threshold 406 and/or the nonshockable threshold 408 based on whether the ECG of the individual previously exhibited high-amplitude VF within a particular time period, whether the individual is a child or an adult, a non-ECG physiological parameter of the individual, whether the individual has exhibited a pulse within a particular time period, whether chest compressions have been administered during a pause period, whether steepnesses of slopes in the ECG have decreased over time, based on a range and/or trend of shock indices corresponding to previous segments of the ECG, or a combination thereof.

The adjustment to the shockable threshold 406 and/or the nonshockable threshold 408 is symmetric or asymmetric. For example, in some cases, the medical device adjusts both of the shockable threshold 406 and the nonshockable threshold 408 symmetrically, such that any increase in the shockable threshold 406 corresponds to a decrease in the nonshockable threshold 408, or vice versa. For example, if the individual is a child, the medical device may decrease the shockable threshold 406 and increase the nonshockable threshold 408 symmetrically. Similarly, if the medical device determines that chest compressions have been previously administered during a pause period, the medical device may decrease the shockable threshold 406 and increase the nonshockable threshold 408 symmetrically. In some cases, the medical device adjusts the shockable threshold 406 and/or the nonshockable threshold 408 asymmetrically, such that any increase in the shockable threshold 406 is asymmetric with any decrease, if any, in the nonshockable threshold 408, or vice versa. An asymmetric adjustment in the shockable threshold 406 and the nonshockable threshold 408 is appropriate when the medical device concludes, based on an analysis factor, that a certainty of the shockable decision should be different than a certainty of the nonshockable decision. For instance, if the medical device determines that the individual previously exhibited high-amplitude VF, the medical device may asymmetrically increase the nonshockable threshold 408.

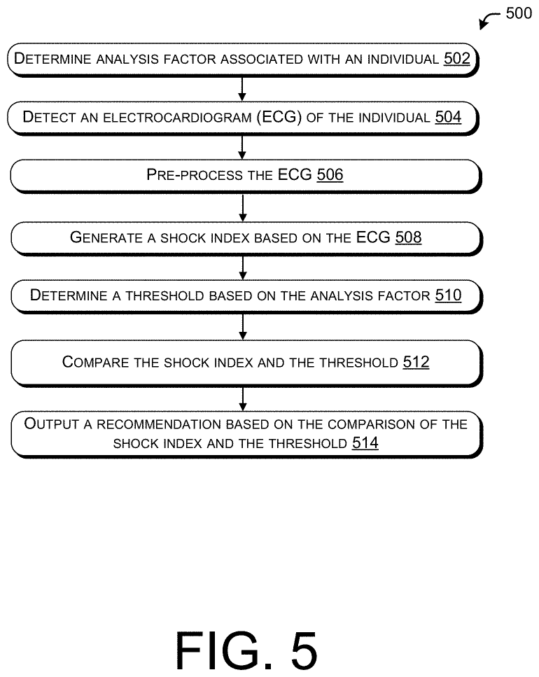

FIGS. 5 to 7 illustrate processes in accordance with various implementations of the present disclosure. Although the processes in FIGS. 5 to 7 are illustrated in particular orders, implementations of the present disclosure are not necessarily limited to the particular orders depicted in FIGS. 5 to 7.

FIG. 5 illustrates an example process 500 for determining a threshold based on an analysis factor and using the threshold to output a recommendation indicating whether a defibrillation shock is advised. The process 500 is performed by a medical device, such as the monitor-defibrillator 102 discussed above with reference to FIG. 1.

At 502, the medical device determines an analysis factor associated with an individual. In some instances, the analysis factor is based on a previous heart rhythm exhibited by the individual. For example, the medical device identifies a segment of an ECG of the individual and determines whether the segment includes VF. If the segment includes VF, the medical device further determines whether the VF is high-amplitude VF by comparing an amplitude (e.g., an average peak amplitude) of the segment to a threshold. In examples in which the segment is detected from the individual while the individual is receiving chest compressions, the medical device removes a chest compression artifact from the segment before determining whether the segment includes VF. In some cases, the medical device determines that the individual exhibits VF based on a shock index of the segment, a frequency of the segment, or a combination thereof. The analysis factor is based on whether the high-amplitude VF has been detected within a particular time period, such as a time period between the previous five minutes and the previous 1 minute. For example, the analysis factor is based on whether the high-amplitude VF has been detected within the past 2 minutes.

In some examples, the analysis factor is based on whether chest compressions are being administered to the individual manually or by a mechanical chest compression device. For instance, the medical device determines that the chest compressions are administered by the mechanical chest compression device based on a signal received from the mechanical chest compression device. In some cases, the medical device determines that the chest compressions are administered manually or by the mechanical chest compression device based on an input signal received from a user (e.g., a rescuer).

In some cases, the analysis factor is based on whether the individual is a child or an adult. For example, the medical device that the individual is a child or an adult based on an input signal received from a user.

In various examples, the analysis factor is based on a non-ECG physiological parameter of the individual. In some cases, the medical device includes and/or is communicatively coupled with a parameter sensor that detects the non-ECG physiological parameter. Examples of the non-ECG physiological parameter include, for instance, a blood pressure of the individual, an oximetry level of the individual, a capnography level of the individual, an acceleration of the individual, another physiological parameter indicative of a physiological condition of the individual, or a combination thereof. In some examples, the analysis factor includes whether the individual has exhibited a pulse during a particular time period, such as a time period between the last 5 minutes or the last 1 minute. For instance, the analysis factor is based on whether the individual has exhibited a pulse, or has been pulseless, within the past 2 minutes. The medical device determines whether the individual has exhibited a pulse based on the non-ECG physiological parameter.

In some examples, the analysis factor is based on whether the individual received chest compressions during a previous CPR pause period. For instance, the medical device outputs an instruction to pause chest compressions to the individual during a pause period. However, the medical device detects chest compressions during the pause period.

In some instances, the analysis factor is based on a change in a steepness of the individual's heart rhythm over time. For example, the medical device determines a slope of a first segment of the ECG of the individual and a slope of a second segment of the ECG, wherein the second segment is detected after the first segment. The medical device determines a difference between the slopes. The analysis factor, for example, is based on that difference.

According to some examples, the analysis factor is based on at least one previous shock index of the ECG of the individual. For example, the medical device determines multiple shock indices corresponding to multiple segments of the ECG over time. The analysis factor is based on a shock index of a previously detected segment of the ECG, in some cases. In some implementations, the medical device determines a trend or range of the shock indices, and the analysis factor is based on the trend or range.

At 504, the medical device detects an ECG of the individual. In various examples, the medical device detects a segment of the ECG of the individual while the individual is receiving chest compressions. Due to the chest compressions, the segment of the ECG includes a chest compression artifact.

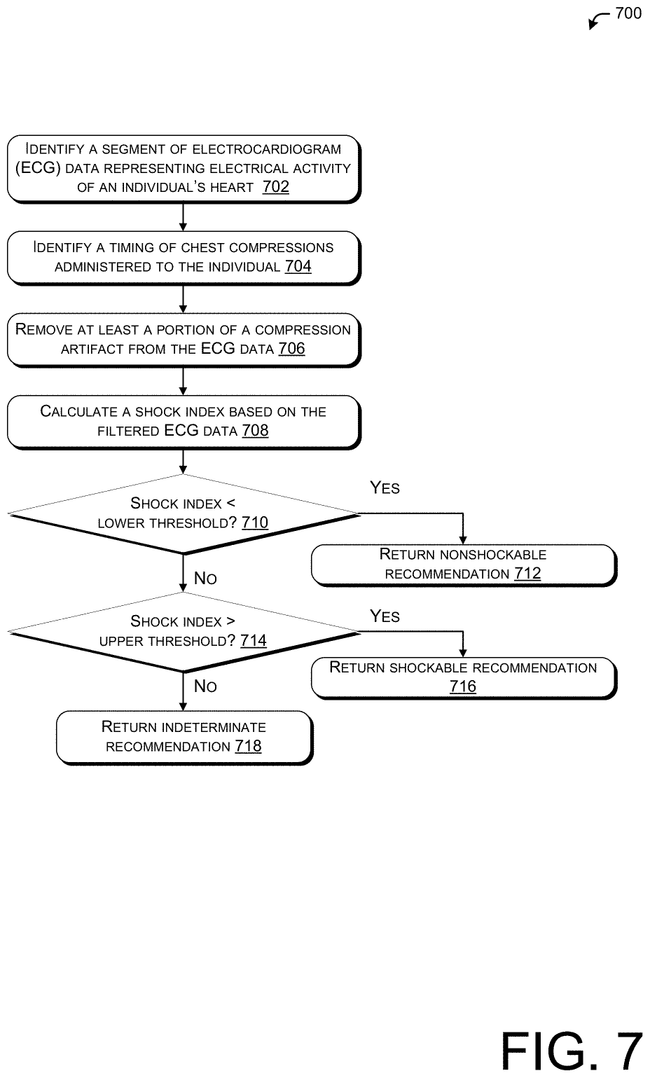

At 506, the medical device pre-processes the ECG. Various examples of techniques for pre-processing the ECG are described below with reference to FIG. 7. For instance, the medical device removes at least a portion of the chest compression artifact from the segment of the ECG. In some cases, the medical device removes at least a portion of the chest compression artifact by applying a filter (e.g., a Kalman filter, an FIR filter, a comb filter, a high-pass filter, or a combination thereof) to the segment.

At 508, the medical device generates a shock index based on the ECG. Various techniques for generating a shock index are described below with reference to FIG. 7. The shock index, for example, is a number indicative of a probability and/or certainty that the segment of the ECG includes a shockable rhythm, such as VF or pulseless V-tach.

At 510, the medical device determines a threshold based on the analysis factor. For instance, the medical device generates and/or adjusts the threshold based on the analysis factor. The threshold corresponds to a threshold shock index. In some examples, the medical device determines the threshold based on whether the individual has exhibited high-amplitude VF in during the time period. For example, if the individual has exhibited high-amplitude VF, then the medical device generates the threshold to be more likely to detect shockable asystole rather than low-amplitude VF.

In various examples, the medical device determines the threshold based on whether the chest compressions are administered by a chest compression device. For instance, if the chest compressions have been administered by the chest compression device, the medical device generates the threshold to be more likely to detect nonshockable asystole or a shockable rhythm rather than a nonshockable rhythm including QRS complexes.

In some cases, the medical device determines the threshold based on whether the individual is a child. For example, the medical device adjusts the threshold to be more likely to come to a shockable or nonshockable decision, rather than an indeterminate decision, if the individual is a child.

According to some implementations, the medical device determines the threshold based on the non-ECG physiological parameter and/or whether the individual has exhibited a pulse during the time period. If the individual has exhibited a pulse during the time period, then the medical device generates the threshold to be more likely to indicate that the individual is exhibiting asystole rather than low-amplitude VF.

In various examples, the medical device determines the threshold based on the change in the steepness of the slopes of the ECG over time. For example, if the individual has exhibited pulseless V-tach and the steepness of the slopes has decreased over time, then the medical device generates the threshold to be more likely to indicate that the individual is continuing to exhibit shockable pulseless V-tach than a nonshockable rhythm.

In some examples, the medical device determines threshold based on the previous shock index exhibited by the individual. For example, if the medical device determines that the individual's previous shock indices were indicative of nonshockable asystole, then the medical device generates the threshold to be less likely to indicate that the individual is exhibiting a shockable rhythm.

At 512, the medical device compares the shock index and the threshold. For example, the medical device determines that the shock index is above the threshold, below the threshold, or equal to the threshold. In various examples, the comparison between the shock index and the threshold is indicative of whether the individual is exhibiting a shockable rhythm, such as VF or pulseless V-tach. In particular instances, the threshold is a shockable threshold and the medical device comes to a shockable decision by determining that the shock index is above the shockable threshold. In particular examples, the threshold is a nonshockable threshold and the medical device comes to a nonshockable decision by determining that the shock index is below the nonshockable threshold. In some cases, the medical device compares the shock index to multiple thresholds. For instance, the medical device comes to an indeterminate decision if the shock index is between the thresholds.

At 514, the medical device outputs a recommendation based on the comparison of the shock index and the threshold. In various examples, the recommendation indicates whether administration of a defibrillation shock to the individual is advised. For example, if the medical device comes to a shockable decision, the recommendation indicates that the defibrillation shock is advised; if the medical device comes to a nonshockable decision, the recommendation indicates the defibrillation shock is not advised; and if the medical device comes to an indeterminate decision, the recommendation indicates the indeterminate decision, that further analysis should be performed, and/or that a CPR pause is advised.

In various implementations, a user of the medical device treats the individual based on the recommendation. For example, the user of the medical device administrates the defibrillation shock if the recommendation indicates that the shock is advised. In some cases, the medical device administrates the defibrillation shock to the individual.

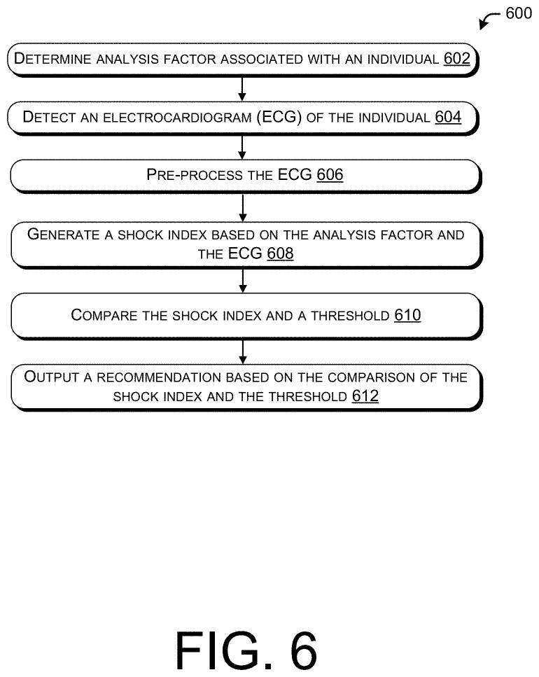

FIG. 6 illustrates an example process 600 for determining a shock index based on an analysis factor and using the shock index to output a recommendation indicating whether a defibrillation shock is advised. The process 600 is performed by a medical device, such as the monitor-defibrillator 102 discussed above with reference to FIG. 1.

At 602, the medical device determines an analysis factor associated with an individual. In some instances, the analysis factor is based on a previously heart rhythm exhibited by the individual. For example, the medical device identifies a segment of an ECG of the individual and determines whether the segment includes VF. If the segment includes VF, the medical device further determines whether the VF is high-amplitude VF by comparing an amplitude (e.g., an average peak amplitude) of the segment to a threshold. In examples in which the segment is detected from the individual while the individual is receiving chest compressions, the medical device removes a chest compression artifact from the segment before determining whether the segment includes VF. In some cases, the medical device determines that the individual exhibits VF based on a shock index of the segment, a frequency of the segment, or a combination thereof. The analysis factor is based on whether the high-amplitude VF has been detected within a particular time period, such as a time period between the previous five minutes and the previous 1 minute. For example, the analysis factor is based on whether the high-amplitude VF has been detected within the past 2 minutes.

In some examples, the analysis factor is based on whether chest compressions are being administered to the individual manually or by a mechanical chest compression device. For instance, the medical device determines that the chest compressions are administered by the mechanical chest compression device based on a signal received from the mechanical chest compression device. In some cases, the medical device determines that the chest compressions are administered manually or by the mechanical chest compression device based on an input signal received from a user (e.g., a rescuer).

In some cases, the analysis factor is based on whether the individual is a child or an adult. For example, the medical device that the individual is a child or an adult based on an input signal received from a user.

In various examples, the analysis factor is based on a non-ECG physiological parameter of the individual. In some cases, the medical device includes and/or is communicatively coupled with a parameter sensor that detects the non-ECG physiological parameter. Examples of the non-ECG physiological parameter include, for instance, a blood pressure of the individual, an oximetry level of the individual, a capnography level of the individual, an acceleration of the individual, another physiological parameter indicative of a physiological condition of the individual, or a combination thereof. In some examples, the analysis factor includes whether the individual has exhibited a pulse during a particular time period, such as a time period between the last 5 minutes or the last 1 minute. For instance, the analysis factor is based on whether the individual has exhibited a pulse, or has been pulseless, within the past 2 minutes. The medical device determines whether the individual has exhibited a pulse based on the non-ECG physiological parameter.

In some examples, the analysis factor is based on whether the individual received chest compressions during a previous CPR pause period. For instance, the medical device outputs an instruction to pause chest compressions to the individual during a pause period. However, the medical device detects chest compressions during the pause period.

In some instances, the analysis factor is based on a change in a steepness of the individual's heart rhythm over time. For example, the medical device determines a slope of a first segment of the ECG of the individual and a slope of a second segment of the ECG, wherein the second segment is detected after the first segment. The medical device determines a difference between the slopes. The analysis factor, for example, is based on that difference.

According to some examples, the analysis factor is based on at least one previous shock index of the ECG of the individual. For example, the medical device determines multiple shock indices corresponding to multiple segments of the ECG over time. The analysis factor is based on a shock index of a previously detected segment of the ECG, in some cases. In some implementations, the medical device determines a trend or range of the shock indices, and the analysis factor is based on the trend or range.

At 604, the medical device detects an ECG of the individual. In various examples, the medical device detects a segment of the ECG of the individual while the individual is receiving chest compressions. Due to the chest compressions, the segment of the ECG includes a chest compression artifact.

At 606, the medical device pre-processes the ECG. Various examples of techniques for pre-processing the ECG are described below with reference to FIG. 7. For instance, the medical device removes at least a portion of the chest compression artifact from the segment of the ECG. In some cases, the medical device removes at least a portion of the chest compression artifact by applying a filter (e.g., a Kalman filter, an FIR filter, a comb filter, a high-pass filter, or a combination thereof) to the segment.

At 608, the medical device generates a shock index based on the analysis factor and the ECG. Various techniques for generating a shock index are described below with reference to FIG. 7. The shock index, for example, is a number indicative of a probability and/or certainty that the segment of the ECG includes a shockable rhythm, such as VF or pulseless V-tach.

In various cases, the medical device determines the shock index based on the analysis factor. For instance, the medical device generates and/or adjusts the shock index based on the analysis factor. The shock index corresponds to a shock index shock index. In some examples, the medical device determines the shock index based on whether the individual has exhibited high-amplitude VF in during the time period. For example, if the individual has exhibited high-amplitude VF, then the medical device generates the shock index to be more likely to detect shockable asystole rather than low-amplitude VF.

In various examples, the medical device determines the shock index based on whether the chest compressions are administered by a chest compression device. For instance, if the chest compressions have been administered by the chest compression device, the medical device generates the shock index to be more likely to detect nonshockable asystole or a shockable rhythm rather than a nonshockable rhythm including QRS complexes.

In some cases, the medical device determines the shock index based on whether the individual is a child. For example, the medical device adjusts the shock index to be more likely to come to a shockable or nonshockable decision, rather than an indeterminate decision, if the individual is a child.

According to some implementations, the medical device determines the shock index based on the non-ECG physiological parameter and/or whether the individual has exhibited a pulse during the time period. If the individual has exhibited a pulse during the time period, then the medical device generates the shock index to be more likely to indicate that the individual is exhibiting asystole rather than low-amplitude VF.

In various examples, the medical device determines the shock index based on the change in the steepness of the slopes of the ECG over time. For example, if the individual has exhibited pulseless V-tach and the steepness of the slopes has decreased over time, then the medical device generates the shock index to be more likely to indicate that the individual is continuing to exhibit shockable pulseless V-tach than a nonshockable rhythm.

In some examples, the medical device determines shock index based on the previous shock index exhibited by the individual. For example, if the medical device determines that the individual's previous shock indices were indicative of nonshockable asystole, then the medical device generates the shock index to be less likely to indicate that the individual is exhibiting a shockable rhythm.

At 610, the medical device compares the shock index and a threshold. the medical device compares the shock index and the threshold. For example, the medical device determines that the shock index is above the threshold, below the threshold, or equal to the threshold. In various examples, the comparison between the shock index and the threshold is indicative of whether the individual is exhibiting a shockable rhythm, such as VF or pulseless V-tach. In particular instances, the threshold is a shockable threshold and the medical device comes to a shockable decision by determining that the shock index is above the shockable threshold. In particular examples, the threshold is a nonshockable threshold and the medical device comes to a nonshockable decision by determining that the shock index is below the nonshockable threshold. In some cases, the medical device compares the shock index to multiple thresholds. For instance, the medical device comes to an indeterminate decision if the shock index is between the thresholds.

At 612, the medical device outputs a recommendation based on the comparison of the shock index and the threshold. In various examples, the recommendation indicates whether administration of a defibrillation shock to the individual is advised. For example, if the medical device comes to a shockable decision, the recommendation indicates that the defibrillation shock is advised; if the medical device comes to a nonshockable decision, the recommendation indicates the defibrillation shock is not advised; and if the medical device comes to an indeterminate decision, the recommendation indicates the indeterminate decision, that further analysis should be performed, and/or that a CPR pause is advised.

In various implementations, a user of the medical device treats the individual based on the recommendation. For example, the user of the medical device administrates the defibrillation shock if the recommendation indicates that the shock is advised. In some cases, the medical device administrates the defibrillation shock to the individual.

FIG. 7 illustrates an example process 700 for identifying a shockable rhythm in ECG data that includes a chest compression artifact. The process 700 is performed by a medical device, such as the monitor-defibrillator 102 described above with reference to FIG. 1 and/or any medical device described above with reference to FIGS. 2-5.

At 702, the medical device identifies a segment of ECG data representing an electrical activity of an individual's heart when the individual is receiving chest compressions. The ECG data is obtained by detecting one or more relative voltages between electrodes connected to the chest of the individual, for instance. The ECG data is digital data representing the detected voltages, for example. According to various implementations, the chest compressions generate artifact in the ECG data. The artifact is at least partly based on jostling or movement of the electrodes on the skin of the individual, for example. An artifact is present in the ECG data based on the chest compressions. If the raw ECG data is output to a user, the chest compression artifact makes the ECG data difficult for the user to evaluate, in some cases. For instance, the user may have difficulty manually discerning whether a shockable rhythm (e.g., VF or pulseless V-Tach) is present in the ECG data. Accordingly, the medical device removes the artifact and automatically determines whether the shockable rhythm is present.

The segment is selected from the ECG data. As used herein, the term “segment” can refer to a subset of data that are obtained from a first time to a second time, wherein the first time occurs after the time of the first datapoint in the data and/or the second time occurs before the time of the last datapoint in the data. In some cases, the data in the segment are obtained over a time interval. The time interval, for example, is at least a minimum period and no longer than a maximum period. The minimum period, for instance, is 3 seconds, 4 seconds, 7 seconds, 10 seconds, or another time interval. The maximum period, for example, is 12 seconds, 20 seconds, 30 seconds, or some other time interval.

At 704, the medical device identifies chest compressions administered to the individual. In some cases, the medical device determines when the chest compressions are administered based on a signal from a chest compression monitor, which in some cases is disposed on the chest of the individual includes at least one accelerometer and/or gyroscope that detects chest compressions administered to the individual. In some examples, the medical device detects an electrical impedance between two or more electrodes in contact with the individual and determines when the chest compressions are administered based on the electrical impedance. The chest compressions are administered to the individual during a time period at which the segment of the ECG data is detected, such that the chest compressions cause the chest compression artifact.

At 706, the medical device generates filtered ECG data by removing the chest compression artifact of the selected segment of the ECG data. The chest compression artifact has a fundamental that is between 1.5 to 2 Hz, in various examples. However, heart rhythm features (e.g., a VF rhythm, a V-tach rhythm, QRS complexes, and other inherent heart rhythms) are typically defined by higher frequencies. In some examples, the medical device applies a filter to the detected ECG segment, such as an adaptive filter (e.g., a Wiener filter, a Kalman filter, or the like), an nth order filter (e.g., a zero-th order filter) a comb filter, an inverse comb filter, a high-pass filter, a band reject filter, a finite impulse response (FIR) filter, an infinite impulse response (IIR) filter, or a combination thereof. In some cases, the medical device converts the ECG segment from the time domain into the frequency (e.g., a Fourier) domain, a Laplace domain, a Z-transform domain, or a wavelet (e.g., a continuous wavelet transform, a discrete wavelet transform, etc.) domain, and removes at least a portion of the chest compression artifact by processing the converted ECG. According to some examples, the medical device identifies and subtracts the chest compression artifact. For instance, the medical device identifies and subtracts the chest compression artifact based on the detected chest compressions. For example, the medical device cross-correlates the ECG segment with data corresponding to the chest compressions (e.g., the impedance, the acceleration of the compression detector, the velocity of the compression detector, etc.), identifies the chest compression artifact based on the cross-correlation, and subtracts the chest compression artifact from the ECG segment. In some instances, the medical device denoises the ECG segment. For example, the medical device removes at least a portion of the chest compression artifact by performing spectral subtraction on the ECG segment.

Optionally, the medical device applies additional filtering techniques to reduce the harmonics of the chest compression artifact in the selected segment of the ECG data. For example, the medical device applies a comb filter with multiple stopbands that correspond to the fundamental frequency of the chest compressions administered to the individual and one or more harmonics of the fundamental frequency.

At 708, the medical device calculates a shock index based on the filtered ECG data. The shock index, for example, corresponds to a likelihood that the original ECG data and/or the filtered ECG data exhibits a rhythm that is treatable with defibrillation. For example, the shock index relates to the likelihood that the filtered ECG data is indicative that the individual is exhibiting VF or pulseless V-Tach. In some examples, the medical device calculates the shock index by detecting a shockable rhythm (e.g., VF or pulseless V-Tach) in the filtered ECG data. In some cases, the medical device performs a rules-based analysis on the filtered ECG data. In some examples, the shock index is generated based on an amplitude magnitude spectrum area (AMSA) of the filtered ECG data, an amplitude of the filtered ECG data, a frequency of the filtered ECG data, or a combination thereof. In some implementations, the medical device calculates the shock index by determining a spectral similarity between the filtered ECG and a sample ECG with a known shockable rhythm (e.g., VF or pulseless V-Tach) and/or by determining a spectral dissimilarity between the filtered ECG and a sample ECG with a known nonshockable rhythm (e.g., asystole, a sinus rhythm including QRS complexes, etc.). In some examples the medical device uses non-ECG data to generate the shock index, at least in part. For instance, the medical device generates the shock index based on a non-ECG physiological parameter (e.g., a heart rate level or waveform, a temperature level or waveform, an airway CO₂ level or waveform, an oxygenation level or waveform, a blood pressure level or waveform, etc.) of the individual, a type of equipment monitoring the individual, a demographic of the individual, or a combination thereof. In some examples, the shock index is calculated based on a regression (e.g., linear regression, binary regression, polynomial regression, logistic regression, nonlinear regression, nonparametric regression, etc.) model outputting a probability that the filtered ECG exhibits a shockable rhythm based on one or more characteristics of the filtered ECG. In various implementations, the medical device generates the shock index based on one or more analysis factors.

At 710, the medical device determines whether the shock index is less than a lower threshold. The lower threshold is selected, for instance, based on an acceptable level of uncertainty regarding a nonshockable recommendation. In some cases, the lower threshold is user-selected, such that the lower threshold is calculated based on an input signal from a user. In some cases, the lower threshold is determined based on one or more analysis factors. If the medical device determines that the shock index is less than the lower threshold, the medical device returns a nonshockable recommendation at 712.

If, on the other hand, the medical device determines that the shock index is greater than or equal to the lower threshold, the process 700 proceeds to 714. At 714, the medical device determines whether the shock index is greater than the upper threshold. The upper threshold is selected, for instance, based on an acceptable level of uncertainty regarding a shockable recommendation. In some cases, the upper threshold is user-selected, such that the upper threshold is calculated based on an input signal from a user. In some examples, the upper threshold is determined based on one or more analysis factors. If the medical device determines that the shock index is greater than the upper threshold, the medical device returns a shockable recommendation at 716.

However, if the medical device determines that the shock index is less than or equal to the upper threshold, then the medical device returns an indeterminate recommendation at 718. The indeterminate decision means that the medical device is unable to conclude whether the shockable rhythm is present with a sufficient level of certainty. The level of certainty, in some cases, is predetermined and/or selected by a user.

In various cases, the medical device performs the process 700 repeatedly, periodically, or a combination thereof. For example, upon returning a recommendation, the medical device repeats the process 700 by identifying another segment of ECG data. In some cases, the medical device initiates the process 700 (e.g., begins 702) at a particular frequency, such that the medical device may be performing the process 700 multiple times, in parallel, at a time. If the medical device determines multiple recommendations based on repeatedly and/or periodically performing the process 700, the medical device outputs (e.g., to the user) a recommendation based on the most recently returned shock decision.

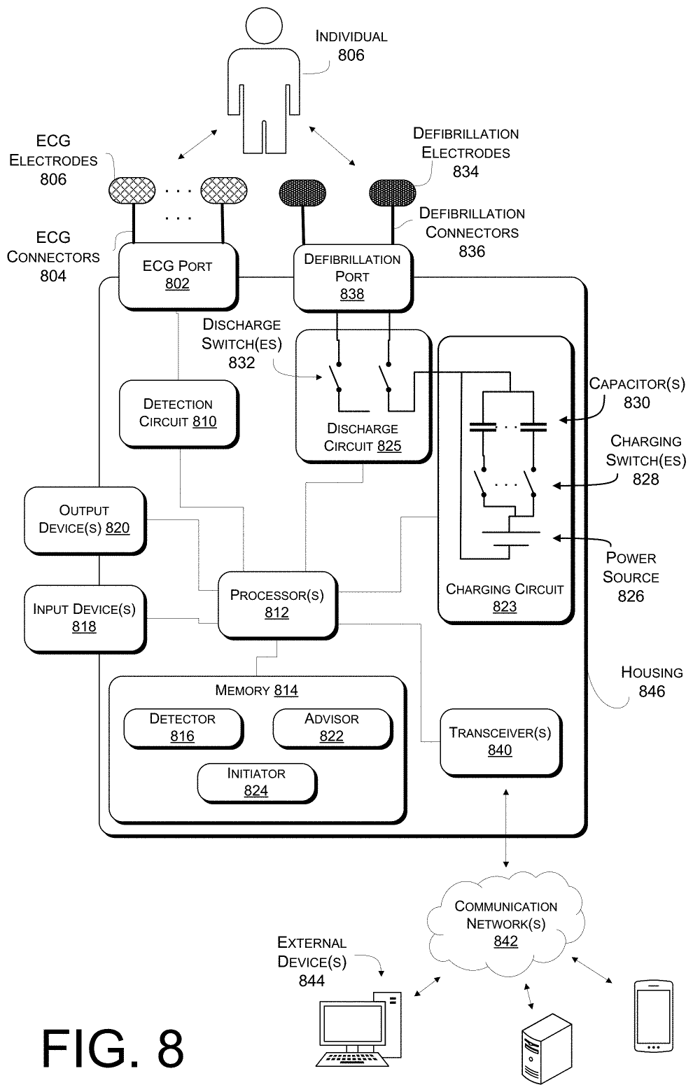

FIG. 8 illustrates an example of an external defibrillator 800 configured to perform various functions described herein. For example, the external defibrillator 800 is the monitor-defibrillator 102 described above with reference to FIG. 1.

The external defibrillator 800 includes an electrocardiogram (ECG) port 802 connected to multiple ECG connectors 804. In some cases, the ECG connectors 804 are removeable from the ECG port 802. For instance, the ECG connectors 804 are plugged into the ECG port 802. The ECG connectors 804 are connected to ECG electrodes 806, respectively. In various implementations, the ECG electrodes 806 are disposed on different locations on an individual 808. A detection circuit 810 is configured to detect relative voltages between the ECG electrodes 806. These voltages are indicative of the electrical activity of the heart of the individual 808.

In various implementations, the ECG electrodes 806 are in contact with the different locations on the skin of the individual 808. In some examples, a first one of the ECG electrodes 806 is placed on the skin between the heart and right arm of the individual 808, a second one of the ECG electrodes 806 is placed on the skin between the heart and left arm of the individual 808, and a third one of the ECG electrodes 806 is placed on the skin between the heart and a leg (either the left leg or the right leg) of the individual 808. In these examples, the detection circuit 808 is configured to measure the relative voltages between the first, second, and third ECG electrodes 806. Respective pairings of the ECG electrodes 806 are referred to as “leads,” and the voltages between the pairs of ECG electrodes 806 are known as “lead voltages.” In some examples, more than three ECG electrodes 806 are included, such that 5-lead or 12-lead ECG signals are detected by the detection circuit 810.

The detection circuit 810 includes at least one analog circuit, at least one digital circuit, or a combination thereof. The detection circuit 810 receives the analog electrical signals from the ECG electrodes 806, via the ECG port 802 and the ECG connectors 804. In some cases, the detection circuit 810 includes one or more analog filters configured to filter noise and/or artifact from the electrical signals. The detection circuit 810 includes an analog-to-digital (ADC) in various examples. The detection circuit 810 generates a digital signal indicative of the analog electrical signals from the ECG electrodes 806. This digital signal can be referred to as an “ECG signal” or an “ECG.”

In some cases, the detection circuit 810 further detects an electrical impedance between at least one pair of the ECG electrodes 806. For example, the detection circuit 810 includes, or otherwise controls, a power source that applies a known voltage across a pair of the ECG electrodes 806 and detects a resultant current between the pair of the ECG electrodes 806. The impedance is generated based on the applied voltage and the resultant current. In various cases, the impedance corresponds to respiration of the individual 808, chest compressions performed on the individual 808, and other physiological states of the individual 808. In various examples, the detection circuit 810 includes one or more analog filters configured to filter noise and/or artifact from the resultant current. The detection circuit 810 generates a digital signal indicative of the impedance using an ADC. This digital signal can be referred to as an “impedance signal” or an “impedance.”

The detection circuit 810 provides the ECG signal and/or the impedance signal one or more processors 812 in the external defibrillator 800. In some implementations, the processor(s) 812 includes a central processing unit (CPU), a graphics processing unit (GPU), both CPU and GPU, or other processing unit or component known in the art.

The processor(s) 812 is operably connected to memory 814. In various implementations, the memory 812 is volatile (such as random access memory (RAM)), non-volatile (such as read only memory (ROM), flash memory, etc.) or some combination of the two. The memory 814 stores instructions that, when executed by the processor(s) 812, causes the processor(s) 812 to perform various operations. In various examples, the memory 814 stores methods, threads, processes, applications, objects, modules, any other sort of executable instruction, or a combination thereof. In some cases, the memory 814 stores files, databases, or a combination thereof. In some examples, the memory 814 includes, but is not limited to, RAM, ROM, electrically erasable programmable read-only memory (EEPROM), flash memory, or any other memory technology. In some examples, the memory 814 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 812 and/or the external defibrillator 800. In some cases, the memory 814 at least temporarily stores the ECG signal and/or the impedance signal.

In various examples, the memory 814 includes a detector 816, which causes the processor(s) 812 to determine, based on the ECG signal and/or the impedance signal, whether the individual 808 is exhibiting a particular heart rhythm. For instance, the processor(s) 812 determines whether the individual 808 is experiencing a shockable rhythm that is treatable by defibrillation. Examples of shockable rhythms include ventricular fibrillation (VF) and pulseless ventricular tachycardia (V-Tach). In some examples, the processor(s) 812 determines whether any of a variety of different rhythms (e.g., asystole, sinus rhythm, atrial fibrillation (AF), etc.) are present in the ECG signal. In various examples, the processor(s) 812 generates a shock index based on the ECG signal, compares the shock index to one or more thresholds, and determines whether the individual 808 is exhibiting the shockable rhythm based on the comparison. In some examples, the processor(s) 812 generates the shock index, the upper threshold, the lower threshold, or a combination thereof, based on an analysis factor. Examples of the analysis factors include, for instance, whether the individual has previously exhibited high-amplitude VF, whether the individual is receiving chest compressions from a mechanical chest compression device, a non-ECG physiological parameter of the individual, whether the individual has previously exhibited a pulse, whether the individual received chest compressions during a CPR pause period, whether a steepness of slopes in the ECG of the individual has decreased over time, a previous shock index of the individual, a range and/or trend of shock indices of the individual, or a combination thereof.

The processor(s) 812 is operably connected to one or more input devices 818 and one or more output devices 820. Collectively, the input device(s) 818 and the output device(s) 820 function as an interface between a user and the defibrillator 800. The input device(s) 818 is configured to receive an input from a user and includes at least one of a keypad, a cursor control, a touch-sensitive display (e.g., a touchscreen), a voice input device (e.g., a speaker), a haptic feedback device, or any combination thereof. The output device(s) 820 includes at least one of a display, a speaker, a haptic output device, a printer, or any combination thereof. In various examples, the processor(s) 812 causes a display among the input device(s) 818 to visually output a waveform of the ECG signal and/or the impedance signal. In some implementations, the input device(s) 818 includes one or more touch sensors, the output device(s) 820 includes a display screen, and the touch sensor(s) are integrated with the display screen. Thus, in some cases, the external defibrillator 800 includes a touchscreen configured to receive user input signal(s) and visually output physiological parameters, such as the ECG signal and/or the impedance signal.

In some examples, the memory 814 includes an advisor 822, which, when executed by the processor(s) 812, causes the processor(s) 812 to generate advice and/or control the output device(s) 820 to output the advice to a user (e.g., a rescuer). In some examples, the processor(s) 812 provides, or causes the output device(s) 820 to provide, an instruction to perform CPR on the individual 808. In some cases, the processor(s) 812 evaluates, based on the ECG signal, the impedance signal, or other physiological parameters, CPR being performed on the individual 808 and causes the output device(s) 820 to provide feedback about the CPR in the instruction. According to some examples, the processor(s) 812, upon identifying that a shockable rhythm is present in the ECG signal, causes the output device(s) 820 to output an instruction and/or recommendation to administer a defibrillation shock to the individual 808.

The memory 814 also includes an initiator 824 which, when executed by the processor(s) 812, causes the processor(s) 812 to control other elements of the external defibrillator 800 in order to administer a defibrillation shock to the individual 808. In some examples, the processor(s) 812 executing the initiator 824 selectively causes the administration of the defibrillation shock based on determining that the individual 808 is exhibiting the shockable rhythm and/or based on an input from a user (received, e.g., by the input device(s) 818. In some cases, the processor(s) 812 causes the defibrillation shock to be output at a particular time, which is determined by the processor(s) 812 based on the ECG signal and/or the impedance signal.

The processor(s) 812 is operably connected to a charging circuit 823 and a discharge circuit 825. In various implementations, the charging circuit 823 includes a power source 826, one or more charging switches 828, and one or more capacitors 830. The power source 826 includes, for instance, a battery. The processor(s) 812 initiates a defibrillation shock by causing the power source 826 to charge at least one capacitor among the capacitor(s) 830. For example, the processor(s) 812 activates at least one of the charging switch(es) 828 in the charging circuit 823 to complete a first circuit connecting the power source 826 and the capacitor to be charged. Then, the processor(s) 812 causes the discharge circuit 825 to discharge energy stored in the charged capacitor across a pair of defibrillation electrodes 830, which are in contact with the individual 808. For example, the processor(s) 812 deactivates the charging switch(es) 828 completing the first circuit between the capacitor(s) 830 and the power source 826, and activates one or more discharge switches 832 completing a second circuit connecting the charged capacitor 830 and at least a portion of the individual 808 disposed between defibrillation electrodes 834. Although not illustrated in FIG. 8, in some implementations, the discharge circuit 825 includes an H-bridge over which the energy from the capacitor(s) 830 is discharged across the defibrillation electrodes 830.

The energy is discharged from the defibrillation electrodes 834 in the form of a defibrillation shock. For example, the defibrillation electrodes 834 are connected to the skin of the individual 808 and located at positions on different sides of the heart of the individual 808, such that the defibrillation shock is applied across the heart of the individual 808. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 832 are controlled by the processor(s) 812, for example. In various implementations, the defibrillation electrodes 834 are connected to defibrillation connectors 836. The defibrillation connectors 836 are connected to a defibrillation port 838, in implementations. According to various examples, the defibrillation connectors 836 are removable from the defibrillation port 838. For example, the defibrillation connectors 836 are plugged into the defibrillation port 838.

In various implementations, the processor(s) 812 is operably connected to one or more transceivers 840 that transmit and/or receive data over one or more communication networks 842. For example, the transceiver(s) 840 includes a network interface card (NIC), a network adapter, a local area network (LAN) adapter, or a physical, virtual, or logical address to connect to the various external devices and/or systems. In various examples, the transceiver(s) 840 includes any sort of wireless transceivers capable of engaging in wireless communication (e.g., radio frequency (RF) communication). For example, the communication network(s) 842 includes one or more wireless networks that include a 3^{rd} Generation Partnership Project (3GPP) network, such as a Long Term Evolution (LTE) radio access network (RAN) (e.g., over one or more LE bands), a New Radio (NR) RAN (e.g., over one or more NR bands), or a combination thereof. In some cases, the transceiver(s) 840 includes other wireless modems, such as a modem for engaging in WI-FI®, WIGIG®, WIMAX®, BLUETOOTH®, or infrared communication over the communication network(s) 842.

The defibrillator 800 is configured to transmit and/or receive data (e.g., ECG data, impedance data, data indicative of one or more detected heart rhythms of the individual 808, data indicative of one or more defibrillation shocks administered to the individual 808, etc.) with one or more external devices 844 via the communication network(s) 842. The external devices 844 include, for instance, mobile devices (e.g., mobile phones, smart watches, etc.), Internet of Things (IoT) devices, medical devices, computers (e.g., laptop devices, servers, etc.), a chest compression device, a parameter sensor, or any other type of computing device configured to communicate over the communication network(s) 842. In some examples, the external device(s) 844 is located remotely from the defibrillator 800, such as at a remote clinical environment (e.g., a hospital). According to various implementations, the processor(s) 812 causes the transceiver(s) 840 to transmit data to the external device(s) 844. In some cases, the transceiver(s) 840 receives data from the external device(s) 844 and the transceiver(s) 840 provide the received data to the processor(s) 812 for further analysis.

In various implementations, the external defibrillator 800 also includes a housing 846 that at least partially encloses other elements of the external defibrillator 800. For example, the housing 846 encloses the detection circuit 810, the processor(s) 812, the memory 814, the charging circuit 823, the transceiver(s) 840, or any combination thereof. In some cases, the input device(s) 818 and output device(s) 820 extend from an interior space at least partially surrounded by the housing 846 through a wall of the housing 846. In various examples, the housing 846 acts as a barrier to moisture, electrical interference, and/or dust, thereby protecting various components in the external defibrillator 800 from damage.

In some implementations, the external defibrillator 800 is an automated external defibrillator (AED) operated by an untrained user (e.g., a bystander, layperson, etc.) and can be operated in an automatic mode. In automatic mode, the processor(s) 812 automatically identifies a rhythm in the ECG signal, makes a decision whether to administer a defibrillation shock, charges the capacitor(s) 830, discharges the capacitor(s) 830, or any combination thereof. In some cases, the processor(s) 812 controls the output device(s) 820 to output (e.g., display) a simplified user interface to the untrained user. For example, the processor(s) 812 refrains from causing the output device(s) 820 to display a waveform of the ECG signal and/or the impedance signal to the untrained user, in order to simplify operation of the external defibrillator 800.

In some examples, the external defibrillator 800 is a monitor-defibrillator utilized by a trained user (e.g., a clinician, an emergency responder, etc.) and can be operated in a manual mode or the automatic mode. When the external defibrillator 800 operates in manual mode, the processor(s) 812 cause the output device(s) 820 to display a variety of information that may be relevant to the trained user, such as waveforms indicating the ECG data and/or impedance data, notifications about detected heart rhythms, and the like.

### Example Clauses

1. An external defibrillator, including: a detection circuit configured to detect an electrocardiogram (ECG) of an individual receiving chest compressions; an output device configured to output a recommendation to administer a defibrillation shock to the individual; a processor; and memory storing instructions that, when executed by the processor, cause the processor to perform operations including: determining that a first segment of the ECG indicates that the individual has ventricular fibrillation (VF), the first segment of the ECG being detected during a first time period; determining whether the VF is coarse VF by comparing an amplitude of the first segment to a first threshold; based on determining whether the VF is coarse VF, generating a second threshold; generating a shock index of a second segment of the ECG, the second segment of the ECG being detected during a second time period occurring after the first time period; determining that the second segment indicates that the individual has VF by determining that the shock index is greater than the second threshold; and based on determining that the second segment indicates that the individual has VF, causing the output device to output the recommendation.
2. The external defibrillator of clause 1, wherein the operations further include: determining whether a mechanical chest compression device is administering the chest compressions to the individual; and determining whether the individual is a child, and wherein generating the second threshold is further based on whether the mechanical chest compression device is administering the chest compressions to the individual and whether the individual is a child.
3. The external defibrillator of clause 1 or 2, further including: a discharge circuit configured to output the defibrillation shock to the individual; an input device configured to receive an input signal from a user, wherein the operations further include: causing the discharge circuit to discharge the defibrillation shock in response to the input device receiving the input signal.
4. A medical device, including a detection circuit configured to detect an electrocardiogram (ECG) of an individual receiving chest compressions; an output device configured to output a recommendation to administer a defibrillation shock to the individual; a processor; and memory storing instructions that, when executed by the processor, cause the processor to perform operations including: determining an analysis factor; determining a threshold based on the analysis factor; generating a filtered segment of the ECG by removing, from the segment, an artifact associated with the chest compressions; generating a shock index based on the filtered segment; determining whether the segment includes a shockable rhythm by comparing the shock index to the threshold; and based on determining whether the segment includes the shockable rhythm, causing the output device to output the recommendation.
5. The medical device of clause 4, the segment of the ECG being a first segment of the ECG detected during a first time period, the threshold being a first threshold, wherein determining the analysis factor includes: identifying a second segment of the ECG detected during a second time period, a start time of the second time period occurring a threshold time or less before a start time of the first time period; determining that the second segment indicates that the individual has ventricular fibrillation (VF); and determining that the VF is high-amplitude VF by comparing an amplitude of the second segment to a second threshold, and wherein determining the threshold based on the analysis factor includes determining the threshold based on determining that the VF in the other segment is high-amplitude VF.
6. The medical device of clause 4 or 5, wherein determining the analysis factor includes determining that the chest compressions are administered by a mechanical chest compression device, and wherein determining the threshold based on the analysis factor includes determining the threshold based on determining that the chest compressions are administered by the mechanical chest compression device.
7. The medical device of any one of clauses 4 to 6, wherein determining the analysis factor includes determining that the individual is a child, and wherein determining the threshold based on the analysis factor includes determining the threshold based on determining that the individual is a child.
8. The medical device of any one of clauses 4 to 7, wherein determining the analysis factor includes identifying a non-ECG physiological parameter of the individual, and wherein determining the threshold based on the analysis factor includes determining the threshold based on the non-ECG physiological parameter.
9. The medical device of clause 8, wherein determining the analysis factor further includes determining, based on the non-ECG physiological parameter, whether the individual has exhibited a pulse within a time period, and wherein determining the threshold based on the analysis factor further includes determining the threshold based on whether the individual has exhibited the pulse within the time period.
10. The medical device of any one of clauses 4 to 9, wherein determining the analysis factor includes determining that at least a portion of the chest compressions were administered to the individual during a cardiopulmonary resuscitation (CPR) period, and wherein determining the threshold based on the analysis factor includes determining the threshold based on determining that at least the portion of the chest compressions were administered to the individual during the CPR period.
11. The medical device of any one of clauses 4 to 10, the segment of the ECG being a first segment of the ECG detected during a first time period, wherein determining the analysis factor includes: determining a first slope of a second segment of the ECG detected during a second time period, an end time of the second time period occurring before a start time of the first time period; determining a second slope of the first segment of the ECG; determining a change between the first slope and the second slope, and wherein determining the threshold based on the analysis factor includes determining the threshold based on the change between the first slope and the second slope.
12. The medical device of any one of clauses 4 to 11, the segment of the ECG being a first segment of the ECG detected during a first time period, wherein determining the analysis factor includes: determining a shock index of a second segment of the ECG detected during a second time period, an end time of the second time period occurring before a start time of the first time period, and wherein determining the threshold based on the analysis factor includes determining the threshold based on the shock index of the second segment of the ECG detected during the second time period.
13. A method performed by a medical device, the method including determining an analysis factor; determining a threshold based on the analysis factor; detecting a segment of an electrocardiogram (ECG) of an individual receiving chest compressions; generating a filtered segment of the ECG by removing, from the segment, an artifact associated with the chest compressions; generating a shock index based on the filtered segment; determining whether the segment includes a shockable rhythm by comparing the shock index to the threshold; and outputting, based on whether the segment includes the shockable rhythm, a recommendation indicating whether a defibrillation shock is advised.
14. The method of clause 13, the segment of the ECG being a first segment of the ECG detected during a first time period, the threshold being a first threshold, wherein determining the analysis factor includes: identifying a second segment of the ECG detected during a second time period, a start time of the second time period occurring a threshold time or less before a start time of the first time period; determining that the second segment indicates that the individual has ventricular fibrillation (VF); and determining that the VF is high-amplitude VF by comparing an amplitude of the second segment to a second threshold, and wherein determining the threshold based on the analysis factor includes determining the threshold based on determining that the VF in the second segment is high-amplitude VF.
15. The method of clause 13 or 14, wherein determining the analysis factor includes determining that the chest compressions are administered by a mechanical chest compression device, and wherein determining the threshold based on the analysis factor includes determining the threshold based on determining that the chest compressions are administered by the mechanical chest compression device.
16. The method of any one of clauses 13 to 15, wherein determining the analysis factor includes determining that the individual is a child, and wherein determining the threshold based on the analysis factor includes determining the threshold based on determining that the individual is a child.
17. The method of any one of clauses 13 to 16, wherein determining the analysis factor includes identifying a non-ECG physiological parameter of the individual, and wherein determining the threshold based on the analysis factor includes determining the threshold based on the non-ECG physiological parameter.
18. The method of any one of clauses 13 to 17, wherein determining the analysis factor includes determining that at least a portion of the chest compressions were administered to the individual during a cardiopulmonary resuscitation (CPR) period, and wherein determining the threshold based on the analysis factor includes determining the threshold based on determining that at least the portion of the chest compressions were administered to the individual during the CPR period.
19. The method of any one of clauses 13 to 18, the segment of the ECG being a first segment of the ECG detected during a first time period, wherein determining the analysis factor includes: determining a first slope of a second segment of the ECG detected during a second time period, an end time of the second time period occurring before a start time of the first time period; determining a second slope of the first segment of the ECG detected during the second time period; determining a change between the first slope and the second slope, and wherein determining the threshold based on the analysis factor includes determining the threshold based on the change between the first slope and the second slope.
20. The method of any one of clauses 13 to 19, the segment of the ECG being a first segment detected during a first time period, wherein determining the analysis factor includes: determining a shock index of a second segment of the ECG detected during a second time period, an end time of the second time period occurring before a start time of the first time period, and wherein determining the threshold based on the analysis factor includes determining the threshold based on the shock index of the second segment of the ECG detected during the second time period.
21. An external defibrillator, including: a detection circuit configured to detect an electrocardiogram (ECG) of an individual receiving chest compressions; an output device configured to output a recommendation to administer a defibrillation shock to the individual; a processor; and memory storing instructions that, when executed by the processor, cause the processor to perform operations including: determining that a first segment of the ECG indicates that the individual has ventricular fibrillation (VF), the first segment of the ECG being detected during a first time period; determining whether the VF is coarse VF by comparing an amplitude of the first segment to a first threshold; based on determining whether the VF is coarse VF, generating a second threshold; generating, based on whether the VF in the first segment of the ECG is coarse VF, a shock index of a second segment of the ECG, the second segment of the ECG being detected during a second time period occurring after the first time period; determining that the second segment indicates that the individual has VF by determining that the shock index is greater than a second threshold; and based on determining that the second segment indicates that the individual has VF, causing the output device to output the recommendation.
22. The external defibrillator of clause 21, wherein the operations further include: determining whether the individual is a child, and wherein generating the shock index of the second segment of the ECG is further based on whether the individual is a child.
23. The external defibrillator of clause 21 or 22, further including: a discharge circuit configured to output the defibrillation shock to the individual; an input device configured to receive an input signal from a user, wherein the operations further include: causing the discharge circuit to discharge the defibrillation shock in response to the input device receiving the input signal.
24. A medical device, including a detection circuit configured to detect an electrocardiogram (ECG) of an individual receiving chest compressions; an output device configured to output a recommendation to administer a defibrillation shock to the individual; a processor; and memory storing instructions that, when executed by the processor, cause the processor to perform operations including: determining an analysis factor; generating a filtered segment of the ECG by removing, from the segment, an artifact associated with the chest compressions; generating a shock index based on the analysis factor and the filtered segment; determining whether the segment includes a shockable rhythm by comparing the shock index to the threshold; and based on determining whether the second segment includes the shockable rhythm, causing the output device to output the recommendation.
25. The medical device of clause 24, the segment of the ECG being a first segment of the ECG detected during a first time period, the threshold being a first threshold, wherein determining the analysis factor includes: identifying a second segment of the ECG detected during a second time period, a start time of the second time period occurring a threshold time or less before a start time of the first time period; determining that the second segment indicates that the individual has ventricular fibrillation (VF); and determining that the VF is high-amplitude VF by comparing an amplitude of the second segment to a second threshold, and wherein generating shock index based on the analysis factor and the filtered segment includes determining the threshold based on determining that the VF in the second segment is high-amplitude VF.
26. The medical device of clause 24 or 25, wherein determining the analysis factor includes determining that the chest compressions are administered by a mechanical chest compression device, and wherein generating shock index based on the analysis factor and the filtered segment includes determining the threshold based on determining that the chest compressions are administered by the mechanical chest compression device.
27. The medical device of any one of clauses 24 to 26, wherein determining the analysis factor includes determining that the individual is a child, and wherein generating shock index based on the analysis factor and the filtered segment includes determining the threshold based on determining that the individual is a child.
28. The medical device of any one of clauses 24 to 27, wherein determining the analysis factor includes identifying a non-ECG physiological parameter of the individual, and wherein generating shock index based on the analysis factor and the filtered segment includes determining the threshold based on the non-ECG physiological parameter.
29. The medical device of clause 28, wherein determining the analysis factor further includes determining, based on the non-ECG physiological parameter, whether the individual has exhibited a pulse within a time period, and wherein generating shock index based on the analysis factor and the filtered segment further includes determining the threshold based on whether the individual has exhibited the pulse within the time period.
30. The medical device of any one of clauses 24 to 29, wherein determining the analysis factor includes determining that at least a portion of the chest compressions were administered to the individual during a cardiopulmonary resuscitation (CPR) period, and wherein generating shock index based on the analysis factor and the filtered segment includes determining the threshold based on determining that at least the portion of the chest compressions were administered to the individual during the CPR period.
31. The medical device of any one of clauses 24 to 30, the segment of the ECG being a first segment of the ECG detected during a first time period, wherein determining the analysis factor includes: determining a first slope of a second segment of the ECG detected during a second time period, an end time of the second time period occurring before a start time of the first time period; determining a second slope of the first segment of the ECG detected during the first time period; determining a change between the first slope and the second slope, and wherein generating shock index based on the analysis factor and the filtered segment includes determining the threshold based on the change between the first slope and the second slope.
32. The medical device of any one of clauses 24 to 31, the segment of the ECG being a first segment of the ECG detected during a first time period, wherein determining the analysis factor includes: determining a shock index of a second segment of the ECG detected during a second time period, an end time of the second time period occurring before a start time of the latter time period, and wherein generating shock index based on the analysis factor and the filtered segment includes determining the threshold based on the shock index of the second segment of the ECG detected during the second time period.
33. A method performed by a medical device, the method including determining an analysis factor; determining a threshold based on the analysis factor; detecting a segment of an electrocardiogram (ECG) of an individual receiving chest compressions; generating a filtered segment of the ECG by removing, from the segment, an artifact associated with the chest compressions; generating a shock index based on the filtered segment; determining whether the segment includes a shockable rhythm by comparing the shock index to the threshold; and outputting, based on whether the segment includes the shockable rhythm, a recommendation indicating whether a defibrillation shock is advised.
34. The method of clause 33, the segment of the ECG being a first segment of the ECG detected during a first time period, the threshold being a first threshold, wherein determining the analysis factor includes: identifying a second segment of the ECG detected during a second time period, a start time of the second time period occurring a threshold time or less before a start time of the first time period; determining that the second segment indicates that the individual has ventricular fibrillation (VF); and determining that the VF is high-amplitude VF by comparing an amplitude of the second segment to a second threshold, and wherein generating shock index based on the analysis factor and the filtered segment includes determining the threshold based on determining that the VF in the second segment is high-amplitude VF.
35. The method of clause 33 or 34, wherein determining the analysis factor includes determining that the chest compressions are administered by a mechanical chest compression device, and wherein generating shock index based on the analysis factor and the filtered segment includes determining the threshold based on determining that the chest compressions are administered by the mechanical chest compression device.
36. The method of any one of clauses 33 to 35, wherein determining the analysis factor includes determining that the individual is a child, and wherein generating shock index based on the analysis factor and the filtered segment includes determining the threshold based on determining that the individual is a child.
37. The method of any one of clauses 33 to 36, wherein determining the analysis factor includes identifying a non-ECG physiological parameter of the individual, and wherein generating shock index based on the analysis factor and the filtered segment includes determining the threshold based on the non-ECG physiological parameter.
38. The method of any one of clauses 33 to 37, wherein determining the analysis factor includes determining that at least a portion of the chest compressions were administered to the individual during a cardiopulmonary resuscitation (CPR) period, and wherein generating shock index based on the analysis factor and the filtered segment includes determining the threshold based on determining that at least the portion of the chest compressions were administered to the individual during the CPR period.
39. The method of any one of clauses 33 to 38, the segment of the ECG being a first segment of the ECG detected during a first time period, wherein determining the analysis factor includes: determining a first slope of a second segment of the ECG detected during a second time period, an end time of the second time period occurring before a start time of the first time period; determining a second slope of the first segment of the ECG detected during the first time period; determining a change between the first slope and the second slope, and wherein generating shock index based on the analysis factor and the filtered segment includes determining the threshold based on the change between the first slope and the second slope.
40. The method of any one of clauses 33 to 39, the segment of the ECG being a first segment of the ECG detected during a first time period, wherein determining the analysis factor includes: determining a shock index of a second segment of the ECG detected during a second time period, an end time of the second time period occurring before a start time of the first time period, and wherein generating shock index based on the analysis factor and the filtered segment includes determining the threshold based on the shock index of the second segment of the ECG detected during the second time period.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. An external defibrillator, comprising:
a detection circuit configured to detect an electrocardiogram (ECG) of an individual receiving chest compressions;
an output device configured to output a recommendation to administer a defibrillation shock to the individual;
a processor; and
memory storing instructions that, when executed by the processor, cause the processor to perform operations comprising:
determining that a first segment of the ECG indicates that the individual has ventricular fibrillation (VF), the first segment of the ECG being detected during a first time period;
determining whether the VF is coarse VF by comparing an amplitude of the first segment to a first threshold;
based on determining whether the VF is coarse VF, generating a second threshold;
generating a shock index of a second segment of the ECG, the second segment of the ECG being detected during a second time period occurring after the first time period;
determining that the second segment indicates that the individual has VF by determining that the shock index is greater than the second threshold; and
based on determining that the second segment indicates that the individual has VF, causing the output device to output the recommendation.

2. The external defibrillator of claim 1, wherein the operations further comprise:
determining whether a mechanical chest compression device is administering the chest compressions to the individual; and
determining whether the individual is a child, and
wherein generating the second threshold is further based on whether the mechanical chest compression device is administering the chest compressions to the individual and whether the individual is a child.

3. The external defibrillator of claim 1, further comprising:
a discharge circuit configured to output the defibrillation shock to the individual;
an input device configured to receive an input signal from a user,
wherein the operations further comprise:
causing the discharge circuit to discharge the defibrillation shock in response to the input device receiving the input signal.

4. A medical device, comprising
a detection circuit configured to detect an electrocardiogram (ECG) of an individual receiving chest compressions;
an output device configured to output a recommendation to administer a defibrillation shock to the individual;
a processor; and
memory storing instructions that, when executed by the processor, cause the processor to perform operations comprising:
determining an analysis factor;
determining a threshold based on the analysis factor;
generating a filtered segment of the ECG by removing, from the segment, an artifact associated with the chest compressions;
generating a shock index based on the filtered segment;
determining whether the segment comprises a shockable rhythm by comparing the shock index to the threshold; and
based on determining whether the segment comprises the shockable rhythm, causing the output device to output the recommendation.

5. The medical device of claim 4, the segment of the ECG being a first segment of the ECG detected during a first time period, the threshold being a first threshold, wherein determining the analysis factor comprises:
identifying a second segment of the ECG detected during a second time period, a start time of the second time period occurring a threshold time or less before a start time of the first time period;
determining that the second segment indicates that the individual has ventricular fibrillation (VF); and
determining that the VF is high-amplitude VF by comparing an amplitude of the second segment to a second threshold, and
wherein determining the threshold based on the analysis factor comprises determining the threshold based on determining that the VF in the other segment is high-amplitude VF.

6. The medical device of claim 4, wherein determining the analysis factor comprises determining that the chest compressions are administered by a mechanical chest compression device, and
wherein determining the threshold based on the analysis factor comprises determining the threshold based on determining that the chest compressions are administered by the mechanical chest compression device.

7. The medical device of claim 4, wherein determining the analysis factor comprises determining that the individual is a child, and
wherein determining the threshold based on the analysis factor comprises determining the threshold based on determining that the individual is a child.

8. The medical device of claim 4, wherein determining the analysis factor comprises identifying a non-ECG physiological parameter of the individual, and
wherein determining the threshold based on the analysis factor comprises determining the threshold based on the non-ECG physiological parameter.

9. The medical device of claim 8, wherein determining the analysis factor further comprises determining, based on the non-ECG physiological parameter, whether the individual has exhibited a pulse within a time period, and
wherein determining the threshold based on the analysis factor further comprises determining the threshold based on whether the individual has exhibited the pulse within the time period.

10. The medical device of claim 4, wherein determining the analysis factor comprises determining that at least a portion of the chest compressions were administered to the individual during a cardiopulmonary resuscitation (CPR) period, and
wherein determining the threshold based on the analysis factor comprises determining the threshold based on determining that at least the portion of the chest compressions were administered to the individual during the CPR period.

11. The medical device of claim 4, the segment of the ECG being a first segment of the ECG detected during a first time period, wherein determining the analysis factor comprises:
determining a first slope of a second segment of the ECG detected during a second time period, an end time of the second time period occurring before a start time of the first time period;
determining a second slope of the first segment of the ECG;
determining a change between the first slope and the second slope, and
wherein determining the threshold based on the analysis factor comprises determining the threshold based on the change between the first slope and the second slope.

12. The medical device of claim 4, the segment of the ECG being a first segment of the ECG detected during a first time period, wherein determining the analysis factor comprises:
determining a shock index of a second segment of the ECG detected during a second time period, an end time of the second time period occurring before a start time of the first time period, and
wherein determining the threshold based on the analysis factor comprises determining the threshold based on the shock index of the second segment of the ECG detected during the second time period.

13. A method performed by a medical device, the method comprising
determining an analysis factor;
determining a threshold based on the analysis factor;
detecting a segment of an electrocardiogram (ECG) of an individual receiving chest compressions;
generating a filtered segment of the ECG by removing, from the segment, an artifact associated with the chest compressions;
generating a shock index based on the filtered segment;
determining whether the segment comprises a shockable rhythm by comparing the shock index to the threshold; and
outputting, based on whether the segment comprises the shockable rhythm, a recommendation indicating whether a defibrillation shock is advised.

14. The method of claim 13, the segment of the ECG being a first segment of the ECG detected during a first time period, the threshold being a first threshold, wherein determining the analysis factor comprises:
identifying a second segment of the ECG detected during a second time period, a start time of the second time period occurring a threshold time or less before a start time of the first time period;
determining that the second segment indicates that the individual has ventricular fibrillation (VF); and
determining that the VF is high-amplitude VF by comparing an amplitude of the second segment to a second threshold, and
wherein determining the threshold based on the analysis factor comprises determining the threshold based on determining that the VF in the second segment is high-amplitude VF.

15. The method of claim 13, wherein determining the analysis factor comprises determining that the chest compressions are administered by a mechanical chest compression device, and
wherein determining the threshold based on the analysis factor comprises determining the threshold based on determining that the chest compressions are administered by the mechanical chest compression device.

16. The method of claim 13, wherein determining the analysis factor comprises determining that the individual is a child, and
wherein determining the threshold based on the analysis factor comprises determining the threshold based on determining that the individual is a child.

17. The method of claim 13, wherein determining the analysis factor comprises identifying a non-ECG physiological parameter of the individual, and
wherein determining the threshold based on the analysis factor comprises determining the threshold based on the non-ECG physiological parameter.

18. The method of claim 13, wherein determining the analysis factor comprises determining that at least a portion of the chest compressions were administered to the individual during a cardiopulmonary resuscitation (CPR) period, and
wherein determining the threshold based on the analysis factor comprises determining the threshold based on determining that at least the portion of the chest compressions were administered to the individual during the CPR period.

19. The method of claim 13, the segment of the ECG being a first segment of the ECG detected during a first time period, wherein determining the analysis factor comprises:
determining a first slope of a second segment of the ECG detected during a second time period, an end time of the second time period occurring before a start time of the first time period;
determining a second slope of the first segment of the ECG detected during the second time period;
determining a change between the first slope and the second slope, and
wherein determining the threshold based on the analysis factor comprises determining the threshold based on the change between the first slope and the second slope.

20. The method of claim 13, the segment of the ECG being a first segment detected during a first time period, wherein determining the analysis factor comprises:
determining a shock index of a second segment of the ECG detected during a second time period, an end time of the second time period occurring before a start time of the first time period, and
wherein determining the threshold based on the analysis factor comprises determining the threshold based on the shock index of the second segment of the ECG detected during the second time period.
